(19) United States
(12) Patent Application Publication
(10) Pub. No.: US 2026/0273311 A1
(43) Pub. Date: Sep. 17, 2026

(54) REAL-TIME MONITORING OF HISTOTRIPSY DOSE DELIVERY DAMAGE SPATIALLY AND TEMPORALLY

(71) Applicant: THE REGENTS OF THE UNIVERSITY OF MICHIGAN, Ann Arbor, MI (US)

(72) Inventors: Zhen XU, Ann Arbor, MI (US); Jonathan R. SUKOVICH, Ann Arbor, MI (US); Timothy L. HALL, Ann Arbor, MI (US); Scott HASKELL, Ann Arbor, MI (US)

(21) Appl. No.: 19/471,961

(22) PCT Filed: Apr. 3, 2024
(86) PCT No.: PCT/US2024/022877

§ 371 Date: Oct. 2, 2025

Related U.S. Application Data
(60) Provisional application No. 63/493,886, filed on Apr. 3, 2023.
(60) Provisional application No. 63/493,889, filed on Apr. 3, 2023.
(60) Provisional application No. 63/493,891, filed on Apr. 3, 2023.

(51) Int. Cl. A61N 7/00

(52) U.S. Cl. CPC A61N 7/00; A61N 2007/0021; A61N 2007/0039; A61N 2007/0078

(57) ABSTRACT

A histotripsy therapy system configured for the treatment of tissue is provided, which may include any number of features. Provided herein are systems and methods that provide efficacious non-invasive and minimally invasive therapeutic, diagnostic and research procedures. Systems and methods are provided which facilitate real-time monitoring of dose delivered and tissue ablation. The techniques can include monitoring received acoustic cavitation emission signals or using dMRI.

### PRIORITY CLAIM

This patent application claims priority to U.S. provisional patent application No. 63/493,886, titled “REAL-TIME MONITORING OF HISTOTRIPSY DOSE DELIVERY DAMAGE SPATIALLY AND TEMPORALLY,” and filed on Apr. 3, 2023, and U.S. provisional patent application No. 63/493,889, titled “HISTOTRIPSY SYSTEMS AND METHODS FOR SELECTIVE ABLATION OF WHITE MATTER IN THE BRAIN,” and filed on Apr. 3, 2023, and U.S. provisional patent application No. 63/493,891, titled “HISTOTRIPSY SYSTEMS AND METHODS FOR FAT REDUCTION,” and filed on Apr. 3, 2023, each of which are herein incorporated by reference in their entirety.

### STATEMENT AS TO FEDERALLY SPONSORED RESEARCH

This invention was made with Government support under Grant Nos. R01-EB032772, R01-EB028309, and R01-CA211217, awarded by the National Institutes of Health. The Government has certain rights in the invention.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present disclosure details novel high intensity therapeutic ultrasound (HITU) systems configured to produce acoustic cavitation, methods, devices and procedures for the minimally and non-invasive treatment of healthy, diseased and/or injured tissue. The acoustic cavitation systems and methods described herein, also referred to Histotripsy, may include transducers, drive electronics, positioning robotics, imaging systems, and integrated treatment planning and control software to provide comprehensive treatment and therapy for soft tissues in a patient.

### BACKGROUND

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, Histotripsy realm; it is necessary to deliver acoustic energy in the form of high amplitude acoustic pulses with low duty cycle.

Compared with conventional focused ultrasound technologies, Histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypoechoic) on ultrasound imaging, so that the operator knows what has been treated; and 4) Histotripsy produces lesions in a controlled and precise manner. It is important to emphasize that unlike thermal ablative technologies such as microwave, radiofrequency, high-intensity focused ultrasound (HIFU) cryo or radiation, Histotripsy relies on the mechanical action of cavitation for tissue destruction and not on heat, cold or ionizing energy.

### SUMMARY OF THE DISCLOSURE

A method of using a transmit-receive histotripsy system for histotripsy treatment monitoring is provided, comprising the steps of: transmitting high-voltage histotripsy therapy pulses into a focal location within a target tissue with transmit electronics and a histotripsy therapy transducer array to generate cavitation in the focal location; receiving low-voltage acoustic cavitation emission (ACE) signals from the cavitation with receive electronics and the histotripsy therapy transducer array; processing the received ACE signals to identify a cavitation lifespan of the cavitation at the focal location; and identifying a plateau in the cavitation lifespan to determine that complete cellular disruption has occurred at the focal location.

In some aspects, the method further comprises generating a 3D map of cavitation produced by the transmitted pulses in real-time.

In some aspects, identifying the plateau comprises identifying a time until a cavitation lifespan plateaus.

In other aspects, identifying the plateau comprises identifying a number of pulses until a cavitation lifespan plateaus.

In some aspects, identifying the plateau comprises identifying when the cavitation lifespan stops increasing.

In one aspect, the method includes automatically stopping transmitting high-voltage histotripsy therapy pulses when it has been determined that complete cellular disruption has occurred at the focal location.

In some aspects, the method includes providing an indication to a user that complete cellular disruption has occurred at the focal location.

In some aspects, the indication comprises an audible alert.

In other aspects, the indication comprises a visual alert.

A histotripsy system is provided, comprising: an ultrasound transducer array;
transmission electronics coupled to the ultrasound transducer array and configured to transmit one or more histotripsy pulses to one or more focal locations generate cavitation in a target tissue; receive electronics configured to receive acoustic cavitation emissions (ACE) from the cavitation; and one or more processors operatively coupled to the transmission and receive electronics, the one or more processors being configured to process the received ACE signals to identify a cavitation lifespan of the cavitation at the focal location and being further configured to identify a plateau in the cavitation lifespan to determine that complete cellular disruption has occurred at the focal location.

In some aspects, the one or more processors are further configured to generate a 3D map of cavitation produced by the transmitted pulses in real-time.

In one aspect, the system includes a display configured to display the 3D map.

In some aspects, the one or more processors are configured to identify the plateau by identifying a time until a cavitation lifespan plateaus.

In other aspects, the one or more processors are configured to identify the plateau by identifying a number of pulses until a cavitation lifespan plateaus.

In some aspects, the one or more processors are configured to identify the plateau by identifying when the cavitation lifespan stops increasing.

In one aspect, the one or more processors are configured to stop transmitting high-voltage histotripsy therapy pulses when it has been determined that complete cellular disruption has occurred at the focal location.

In some aspects, the one or more processors are configured to provide an indication to a user that complete cellular disruption has occurred at the focal location.

In one aspect, the indication comprises an audible alert.

In other aspects, the indication comprises a visual alert.

A method of monitoring histotripsy treatment is provided, comprising the steps of: transmitting high-voltage histotripsy therapy pulses into one or more focal locations within a target tissue with transmit electronics and a histotripsy therapy transducer array to generate cavitation in the focal location; obtaining periodic diffusion weighted magnetic resonance imaging (dMRI) images of the one or more focal locations in the target tissue; processing the dMRI images to identify apparent diffusion coefficients (ADC) in the one or more focal locations in the target tissue; and generating an ADC map that conveys a dose of histotripsy therapy received at each of the one or more focal locations in the target tissue.

In one aspect, the method includes displaying the ADC map.

In other aspects, the method includes increasing ADC in the target tissue indicates increased histotripsy dose received.

In some aspects, the method includes identifying complete cellular disruption at one or more focal locations in the target tissue.

A histotripsy system is provided, comprising: an ultrasound transducer array;

transmission electronics coupled to the ultrasound transducer array and configured to transmit one or more histotripsy pulses to one or more focal locations generate cavitation in a target tissue; a magnetic resonance imaging (MRI) system configured to periodically obtain diffusion weighted magnetic resonance imaging (dMRI) images of the one or more focal locations in the target tissue; and one or more processors operatively coupled to the transmission electronics and the MRI system, the one or more processors being configured to process the dMRI images to identify apparent diffusion coefficients (ADC) in the one or more focal locations in the target tissue, the one or more processors being further configured to generate an ADC map that conveys a dose of histotripsy therapy received at each of the one or more focal locations in the target tissue.

In some aspects, the system includes a display configured to display the ADC map.

In one aspect, increasing ADC in the target tissue indicates increased histotripsy dose received.

In another aspect, the system is configured to identify complete cellular disruption at one or more focal locations in the target tissue.

A method of using a transmit-receive histotripsy system for tissue-type detection is provided, comprising the steps of: transmitting high-voltage histotripsy therapy pulses into one or more focal locations within a target tissue with transmit electronics and a histotripsy therapy transducer array to generate cavitation in the target tissue; receiving low-voltage acoustic cavitation emission (ACE) signals from the cavitation with receive electronics and the histotripsy therapy transducer array; processing the received acoustic cavitation emission signals to identify features relevant to tissue-type; and determining that first tissue at a first focal location has a different tissue type than second tissue at a second focal location based on the identified features.

In one aspect, the method includes generating a 3D map of cavitation produced by the transmitted pulses in real-time.

In some aspects, the identified features comprise a maximal cavitation lifespan at each of the one or more focal locations.

In one aspect, the identified features comprise a time until a cavitation lifespan plateaus.

In some aspects, the identified features comprise a number of pulses until a cavitation lifespan plateaus.

In other aspects, the maximal cavitation lifespan at the first focal location is substantially lower than the maximal cavitation lifespan at the second focal location.

In one aspect, the maximal cavitation lifespan at the first focal location is 2-3× lower than the maximal cavitation lifespan at the second focal location.

In some aspects, the method includes determining that the first tissue comprises a fibrous tissue and the second tissue comprises a cellular tissue.

A histotripsy method is provided, comprising: transmitting histotripsy pulses with a transmit-receive histotripsy transducer array to a first focal location to generate cavitation; receiving ACE signals from the first focal location with the transmit-receive histotripsy transducer; identifying a first maximal cavitation lifespan at the first focal location; mechanically moving or electronically steering the histotripsy therapy transducer array from the first focal location to a second focal location; transmitting histotripsy pulses with the transmit-receive histotripsy transducer array to the second focal location to generate cavitation; receiving ACE signals from the second focal location with the transmit-receive histotripsy transducer, identifying a second maximal cavitation lifespan at the second focal location; and comparing the first maximal cavitation lifespan to the second maximal cavitation lifespan to determine if the second focal location is positioned in a different tissue type than the first focal location.

In some aspects, the method includes determining that the second focal location is in a different tissue type if the second maximal cavitation lifespan is substantially different than the first maximal cavitation lifespan.

In one aspect, the method includes determining that the second focal location is in a different tissue type if the maximal cavitation lifespan at the first focal location is 2-3× lower than the maximal cavitation lifespan at the second focal location.

In some aspects, the method includes determining that the second focal location is in a different tissue type if the maximal cavitation lifespan at the first focal location is 2-3× higher than the maximal cavitation lifespan at the second focal location.

A histotripsy method is provided, comprising: transmitting histotripsy test pulses into one or more test locations within a target tissue with transmit electronics and a histotripsy therapy transducer array to generate cavitation in the target tissue; receiving low-voltage acoustic cavitation emission (ACE) signals from the cavitation at each of the one or more test locations with receive electronics and the histotripsy therapy transducer array; processing the received acoustic cavitation emission signals to determine a tissue-type at the one or more test locations; and modifying a histotripsy treatment plan based on the tissue-type determination to deliver histotripsy therapy to one or more focal locations within a tissue-type to be treated and avoid delivering histotripsy pulses to any location within a tissue-type not to be treated.

A transmit-receive driving electronics of a histotripsy system is provided, comprising: an ultrasound transducer array; transmission electronics coupled to the ultrasound transducer array and configured to transmit one or more histotripsy pulses to one or more focal locations generate cavitation in a target tissue; receive electronics configured to receive acoustic cavitation emissions (ACE) from the cavitation; and one or more processors operatively coupled to the transmission and receive electronics, the one or more processors being configured to process the received ACE signals to identify features relevant to tissue-type and determine a tissue-type at the one or more focal locations based on the identified features.

A histotripsy method is provided, comprising: acoustically coupling a histotripsy therapy transducer to skin of a subject; positioning a focus of the histotripsy therapy transducer within a layer of fat below the skin; and transmitting histotripsy pulses with a peak negative pressure above 14 MPa and below 26 MPa to non-invasively and selectively liquefy the fat and not surrounding tissues.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1B illustrate an ultrasound imaging and therapy system.

FIG. 2 is a system diagram showing a histotripsy therapy system that includes a therapy cart, a fluidics cart, and a coupling system.

FIGS. 3A-3D and 4A-4C show various driving and receiving electronics for enabling a transmit-receive transducer array.

FIGS. 5A-5B show cavitation lifespan measured from ACE in brain tissue vs number of histotripsy pulses and the location within the tissue.

FIGS. 6A-6B show one example of measuring ACE signals resulting from cavitation generated in brain tissue.

FIG. 7 is a method for using ACE signals to determine tissue type at a focal location.

FIGS. 8A-8F show probability data and fit curves for cavitation generation in various sample types.

FIG. 9 shows a technique for selectively ablating fat tissue with histotripsy.

FIG. 10 is a flowchart for selectively ablating fat tissue with histotripsy.

FIGS. 11A-11B show dMRI images of histotripsy lesions in tissue using different doses and the resulting ΔADC value increasing with increasing histotripsy doses.

FIG. 12 is a flowchart for generating ADC maps of tissue treated with histotripsy.

FIG. 13 is a fluidics system for delivering a coupling medium to a coupling container.

### DETAILED DESCRIPTION

The system, methods and devices of the disclosure may be used for open surgical, minimally invasive surgical (laparoscopic and percutaneous), robotic surgical (integrated into a robotically-enabled medical system), endoscopic or completely transdermal extracorporeal non-invasive acoustic cavitation for the treatment of healthy, diseased and/or injured tissue including but not limited to tissue destruction, cutting, skeletonizing and ablation. Furthermore, due to tissue selective properties, histotripsy may be used to create a cytoskeleton that allows for subsequent tissue regeneration either de novo or through the application of stem cells and other adjuvants. Finally, histotripsy can be used to cause the release of delivered agents such as chemotherapy and immunotherapy by locally causing the release of these agents by the application of acoustic energy to the targets. As will be described below, the acoustic cavitation system may include various sub-systems, including a Cart, Therapy, Integrated Imaging, Robotics, Coupling and Software. The system also may comprise various Other Components, Ancillaries and Accessories, including but not limited to computers, cables and connectors, networking devices, power supplies, displays, drawers/storage, doors, wheels, and various simulation and training tools, etc. All systems, methods and means creating/controlling/delivering histotripsy are considered to be a part of this disclosure, including new related inventions disclosed herein.

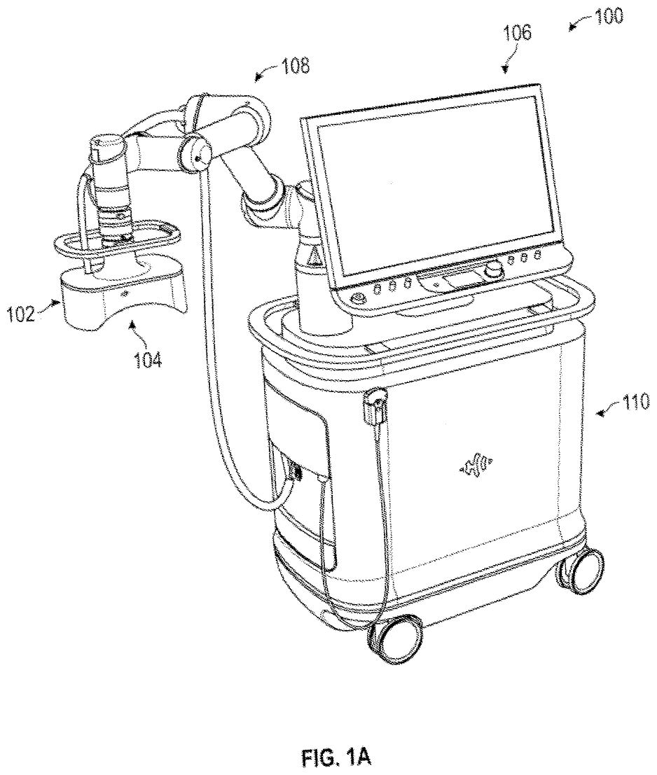

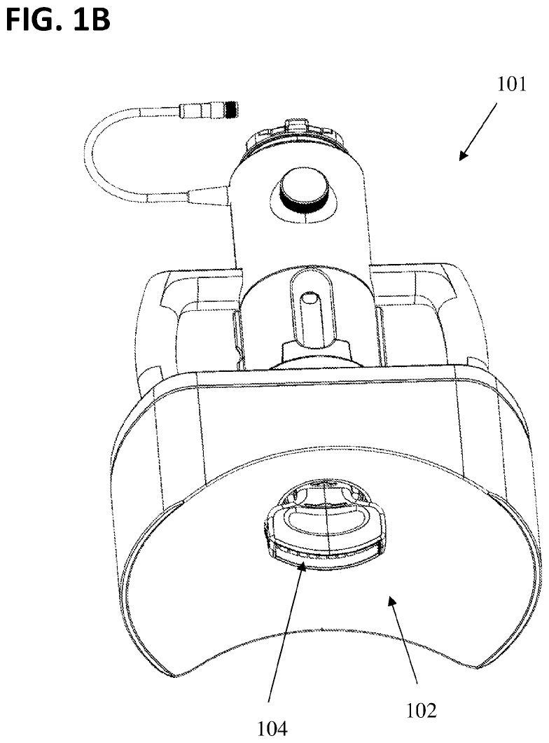

FIG. 1A generally illustrates histotripsy system 100 according to the present disclosure, comprising a therapy transducer 102, an imaging system 104, a display and control panel 106, a robotic positioning arm 108, and a cart 110. The system can further include an ultrasound coupling interface and a source of coupling medium, not shown.

FIG. 1B is a bottom view of the therapy transducer 102 and the imaging system 104. As shown, the imaging system can be positioned in the center of the therapy transducer.

However, other embodiments can include the imaging system positioned in other locations within the therapy transducer, or even directly integrated into the therapy transducer. In some embodiments, the imaging system is configured to produce real-time imaging at a focal point of the therapy transducer. The system also allows for multiple imaging transducers to be located within the therapy transducer to provide multiple views of the target tissue simultaneously and to integrate these images into a single 3-D image.

The histotripsy system may comprise one or more of various sub-systems, including a Therapy sub-system that can create, apply, focus and deliver acoustic cavitation/histotripsy through one or more therapy transducers, Integrated Imaging sub-system (or connectivity to) allowing real-time visualization of the treatment site and histotripsy effect through-out the procedure, a Robotics positioning sub-system to mechanically and/or electronically steer the therapy transducer, further enabled to connect/support or interact with a Coupling sub-system to allow acoustic coupling between the therapy transducer and the patient, and Software to communicate, control and interface with the system and computer-based control systems (and other external systems) and various Other Components, Ancillaries and Accessories, including one or more user interfaces and displays, and related guided work-flows, all working in part or together. The system may further comprise various fluidics and fluid management components, including but not limited to, pumps, valve and flow controls, temperature and degassing controls, and irrigation and aspiration capabilities, as well as providing and storing fluids. It may also contain various power supplies and protectors.

As described above, the histotripsy system may include integrated imaging. However, in other embodiments, the histotripsy system can be configured to interface with separate imaging systems, such as C-arm, fluoroscope, cone beam CT, MRI, etc., to provide real-time imaging during histotripsy therapy. In some embodiments, the histotripsy system can be sized and configured to fit within a C-arm, fluoroscope, cone beam CT, MRI, etc.

### Cart

The Cart 110 may be generally configured in a variety of ways and form factors based on the specific uses and procedures. In some cases, systems may comprise multiple Carts, configured with similar or different arrangements. In some embodiments, the cart may be configured and arranged to be used in a radiology environment and in some cases in concert with imaging (e.g., CT, cone beam CT and/or MRI scanning). In other embodiments, it may be arranged for use in an operating room and a sterile environment for open surgical or laparoscopic surgical and endoscopic application, or in a robotically enabled operating room, and used alone, or as part of a surgical robotics procedure wherein a surgical robot conducts specific tasks before, during or after use of the system and delivery of acoustic cavitation/histotripsy. As such and depending on the procedure environment based on the aforementioned embodiments, the cart may be positioned to provide sufficient work-space and access to various anatomical locations on the patient (e.g., torso, abdomen, flank, head and neck, etc.), as well as providing work-space for other systems (e.g., anesthesia cart, laparoscopic tower, surgical robot, endoscope tower, etc.).

The Cart may also work with a patient surface (e.g., table or bed) to allow the patient to be presented and repositioned in a plethora of positions, angles and orientations, including allowing changes to such to be made pre, peri and post-procedurally. It may further comprise the ability to interface and communicate with one or more external imaging or image data management and communication systems, not limited to ultrasound, CT, fluoroscopy, cone beam CT, PET, PET/CT, MRI, optical, ultrasound, and image fusion and or image flow, of one or more modalities, to support the procedures and/or environments of use, including physical/mechanical interoperability (e.g., compatible within cone beam CT work-space for collecting imaging data pre, peri and/or post histotripsy) and to provide access to and display of patient medical data including but not limited to laboratory and historical medical record data.

In some embodiments one or more Carts may be configured to work together. As an example, one Cart may comprise a bedside mobile Cart equipped with one or more Robotic arms enabled with a Therapy transducer, and Therapy generator/amplifier, etc., while a companion cart working in concert and at a distance of the patient may comprise Integrated Imaging and a console/display for controlling the Robotic and Therapy facets, analogous to a surgical robot and master/slave configurations.

In some embodiments, the system may comprise a plurality of Carts, all slave to one master Cart, equipped to conduct acoustic cavitation procedures. In some arrangements and cases, one Cart configuration may allow for storage of specific sub-systems at a distance reducing operating room clutter, while another in concert Cart may comprise essentially bedside sub-systems and componentry (e.g., delivery system and therapy).

One can envision a plethora of permutations and configurations of Cart design, and these examples are in no way limiting the scope of the disclosure.

### Histotripsy

Histotripsy comprises short, high amplitude, focused ultrasound pulses to generate a dense, energetic, “bubble cloud”, capable of the targeted fractionation and destruction of tissue. Histotripsy is capable of creating controlled tissue erosion when directed at a tissue interface, including tissue/fluid interfaces, as well as well-demarcated tissue fractionation and destruction, at sub-cellular levels, when it is targeted at bulk tissue. Unlike other forms of ablation, including thermal and radiation-based modalities, histotripsy does not rely on heat cold or ionizing (high) energy to treat tissue. Instead, histotripsy uses acoustic cavitation generated at the focus to mechanically effect tissue structure, and in some cases liquefy, suspend, solubilize and/or destruct tissue into sub-cellular components.

Histotripsy can be applied in various forms, including: 1) Intrinsic-Threshold Histotripsy: Delivers pulses with a 1-2 cycles of high amplitude negative/tensile phase pressure exceeding the intrinsic threshold to generate cavitation in the medium (e.g., ~24-28 MPa for water-based soft tissue), 2) Shock-Scattering Histotripsy: Delivers typically pulses 3-20 cycles in duration. The shockwave (positive/compressive phase) scattered from an initial individual microbubble generated forms inverted shockwave, which constructively interfere with the incoming negative/tensile phase to form high amplitude negative/rarefactional phase exceeding the intrinsic threshold. In this way, a cluster of cavitation microbubbles is generated. The amplitude of the tensile phases of the pulses is sufficient to cause bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) Boiling Histotripsy: Employs pulses roughly 1-20 ms in duration. Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer over the skin of a patient and transmitting acoustic pulses transcutaneously through overlying (and intervening) tissue to the focal zone (treatment zone and site). The application of histotripsy is not limited to a transdermal approach but can be applied through any means that allows contact of the transducer with tissue including open surgical laparoscopic surgical, percutaneous and robotically mediated surgical procedures. It may be further targeted, planned, directed and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism”.

This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface, which grows towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without at least one intense shock front developed by nonlinear propagation, no dense bubble clouds are generated when the peak negative half-cycles are below the intrinsic threshold.

When ultrasound pulses less than 2 cycles are applied, shock scattering can be minimized, and the generation of a dense bubble cloud depends on the negative half cycle(s) of the applied ultrasound pulses exceeding an “intrinsic threshold” of the medium. This is referred to as the “intrinsic threshold mechanism”.

This threshold can be in the range of 26-30 MPa for soft tissues with high water content, such as tissues in the human body. In some embodiments, using this intrinsic threshold mechanism, the spatial extent of the lesion may be well-defined and more predictable. With peak negative pressures (P−) not significantly higher than this threshold, sub-wavelength reproducible lesions as small as half of the −6 dB beam width of a transducer may be generated.

With high-frequency Histotripsy pulses, the size of the smallest reproducible lesion becomes smaller, which is beneficial in applications that require precise lesion generation. However, high-frequency pulses are more susceptible to attenuation and aberration, rendering problematical treatments at a larger penetration depth (e.g., ablation deep in the body) or through a highly aberrative medium (e.g., transcranial procedures, or procedures in which the pulses are transmitted through bone(s)). Histotripsy may further also be applied as a low-frequency “pump” pulse (typically <2 cycles and having a frequency between 100 kHz and 1 MHz) can be applied together with a high-frequency “probe” pulse (typically <2 cycles and having a frequency greater than 2 MHz, or ranging between 2 MHz and 10 MHz) wherein the peak negative pressures of the low and high-frequency pulses constructively interfere to exceed the intrinsic threshold in the target tissue or medium. The low-frequency pulse, which is more resistant to attenuation and aberration, can raise the peak negative pressure P-level for a region of interest (ROI), while the high-frequency pulse, which provides more precision, can pin-point a targeted location within the ROI and raise the peak negative pressure P-above the intrinsic threshold. This approach may be referred to as “dual frequency”, “dual beam histotripsy” or “parametric histotripsy.”

Additional systems, methods and parameters to deliver optimized histotripsy, using shock scattering, intrinsic threshold, and various parameters enabling frequency compounding and bubble manipulation, are herein included as part of the system and methods disclosed herein, including additional means of controlling said histotripsy effect as pertains to steering and positioning the focus, and concurrently managing tissue effects (e.g., prefocal thermal collateral damage) at the treatment site or within intervening tissue. Further, it is disclosed that the various systems and methods, which may include a plurality of parameters, such as but not limited to, frequency, operating frequency, center frequency, pulse repetition frequency, pulses, bursts, number of pulses, cycles, length of pulses, amplitude of pulses, pulse period, delays, burst repetition frequency, sets of the former, loops of multiple sets, loops of multiple and/or different sets, sets of loops, and various combinations or permutations of, etc., are included as a part of this disclosure, including future envisioned embodiments of such.

### Therapy Components

The Therapy sub-system may work with other sub-systems to create, optimize, deliver, visualize, monitor and control acoustic cavitation, also referred to herein and in following as “histotripsy”, and its derivatives of, including boiling histotripsy and other thermal high frequency ultrasound approaches. It is noted that the disclosed inventions may also further benefit other acoustic therapies that do not comprise a cavitation, mechanical or histotripsy component. The therapy sub-system can include, among other features, an ultrasound therapy transducer and a pulse generator system configured to deliver ultrasound pulses into tissue. The pulse generator can be incorporated into, for example, the therapy cart such as within cart 110 of FIG. 1A.

In order to create and deliver histotripsy and derivatives of histotripsy, the therapy sub-system may also comprise components, including but not limited to, one or more function generators, amplifiers, therapy transducers and power supplies.

The therapy transducer can comprise a single element or multiple elements configured to be excited with high amplitude electric pulses (>1000V or any other voltage that can cause harm to living organisms). The amplitude necessary to drive the therapy transducers for Histotripsy vary depending on the design of the transducer and the materials used (e.g., solid or polymer/piezoelectric composite including ceramic or single crystal) and the transducer center frequency which is directly proportional to the thickness of the piezoelectric material. Transducers therefore operating at a high frequency require lower voltage to produce a given surface pressure than is required by low frequency therapy transducers. In some embodiments, the transducer elements are formed using a piezoelectric-polymer composite material or a solid piezoelectric material. Further, the piezoelectric material can be of polycrystalline/ceramic or single crystalline formulation. In some embodiments the transducer elements can be formed using silicon using MEMs technology, including CMUT and PMUT designs.

In some embodiments, the function generator may comprise a field programmable gate array (FPGA) or other suitable function generator. The FPGA may be configured with parameters disclosed previously herein, including but not limited to frequency, pulse repetition frequency, bursts, burst numbers, where bursts may comprise pulses, numbers of pulses, length of pulses, pulse period, delays, burst repetition frequency or period, where sets of bursts may comprise a parameter set, where loop sets may comprise various parameter sets, with or without delays, or varied delays, where multiple loop sets may be repeated and/or new loop sets introduced, of varied time delay and independently controlled, and of various combinations and permutations of such, overall and throughout.

In some embodiments, the generator or amplifier may be configured to be a universal single-cycle or multi-cycle pulse generator, and to support driving via Class D or inductive driving, as well as across all envisioned clinical applications, use environments, also discussed in part later in this disclosure. In other embodiments, the class D or inductive current driver may be configured to comprise transformer and/or auto-transformer driving circuits to further provide step up/down components, and in some cases, to preferably allow a step up in the amplitude. They may also comprise specific protective features, to further support the system, and provide capability to protect other parts of the system (e.g., therapy transducer and/or amplifier circuit components) and/or the user, from various hazards, including but not limited to, electrical safety hazards, which may potentially lead to use environment, system and therapy system, and user harms, damage or issues.

Disclosed generators may allow and support the ability of the system to select, vary and control various parameters (through enabled software tools), including, but not limited to those previously disclosed, as well as the ability to start/stop therapy, set and read voltage level, pulse and/or burst repetition frequency, number of cycles, duty ratio, channel enabled and delay, etc., modulate pulse amplitude on a fast time-scale independent of a high voltage supply, and/or other service, diagnostic or treatment features.

In some embodiments, the Therapy sub-system and/or components of, such as the amplifier, may comprise further integrated computer processing capability and may be networked, connected, accessed, and/or be removable/portable, modular, and/or exchangeable between systems, and/or driven/commanded from/by other systems, or in various combinations. Other systems may include other acoustic cavitation/histotripsy, HIFU, HITU, radiation therapy, radiofrequency, microwave, and cryoablation systems, navigation and localization systems, open surgical, laparoscopic, single incision/single port, endoscopic and non-invasive surgical robots, laparoscopic or surgical towers comprising other energy-based or vision systems, surgical system racks or booms, imaging carts, etc.

In some embodiments, one or more amplifiers may comprise a Class D amplifier and related drive circuitry including matching network components. Depending on the transducer element electric impedance and choice of the matching network components (e.g., an LC circuit made of an inductor L1 in series and the capacitor C1 in parallel), the combined impedance can be aggressively set low in order to have high amplitude electric waveform necessary to drive the transducer element. The maximum amplitude that Class D amplifiers is dependent on the circuit components used, including the driving MOSFET/IGBT transistors, matching network components or inductor, and transformer or autotransformer, and of which may be typically in the low kV (e.g., 1-3 kV) range.

Therapy transducer element(s) are excited with an electrical waveform with an amplitude (voltage) to produce a pressure output sufficient for Histotripsy therapy. The excitation electric field can be defined as the necessary waveform voltage per thickness of the piezoelectric element. For example, because a piezoelectric element operating at 1 MHz transducer is half the thickness of an equivalent 500 kHz element, it will require half the voltage to achieve the same electric field and surface pressure.

The Therapy sub-system may also comprise therapy transducers of various designs and working parameters, supporting use in various procedures (and procedure settings). Systems may be configured with one or more therapy transducers, that may be further interchangeable, and work with various aspects of the system in similar or different ways (e.g., may interface to a robotic arm using a common interface and exchange feature, or conversely, may adapt to work differently with application specific imaging probes, where different imaging probes may interface and integrate with a therapy transducer in specifically different ways).

Therapy transducers may be configured of various parameters that may include size, shape (e.g., rectangular or round; anatomically curved housings, etc.), geometry, focal length, number of elements, size of elements, distribution of elements (e.g., number of rings, size of rings for annular patterned transducers), frequency, enabling electronic beam steering, etc. Transducers may be composed of various materials (e.g., piezoelectric, silicon, etc.), form factors and types (e.g., machined elements, chip-based, etc.) and/or by various methods of fabrication of.

Transducers may be designed and optimized for clinical applications (e.g., abdominal tumors, peripheral vascular disease, fat ablation, etc.) and desired outcomes (e.g., acoustic cavitation/histotripsy without thermal injury to intervening tissue), and affording a breadth of working ranges, including relatively shallow and superficial targets (e.g., thyroid or breast nodules), versus, deeper or harder to reach targets, such as central liver or brain tumors. They may be configured to enable acoustic cavitation/histotripsy under various parameters and sets of, as enabled by the aforementioned system components (e.g., function generator and amplifier, etc.), including but not limited to frequency, pulse repetition rate, pulses, number of pulses, pulse length, pulse period, delays, repetitions, sync delays, sync period, sync pulses, sync pulse delays, various loop sets, others, and permutations of. The transducer may also be designed to allow for the activation of a drug payload either deposited in tissue through various means including injection, placement or delivery in micelle or nanostructures.

### Integrated Imaging

The disclosed system may comprise various imaging modalities to allow users to visualize, monitor and collect/use feedback of the patient's anatomy, related regions of interest and treatment/procedure sites, as well as surrounding and intervening tissues to assess, plan and conduct procedures, and adjust treatment parameters as needed. Imaging modalities may comprise various ultrasound, x-ray, CT, MRI, PET, fluoroscopy, optical, contrast or agent enhanced versions, and/or various combinations of. It is further disclosed that various image processing and characterization technologies may also be utilized to afford enhanced visualization and user decision making. These may be selected or commanded manually by the user or in an automated fashion by the system. The system may be configured to allow side by side, toggling, overlays, 3D reconstruction, segmentation, registration, multi-modal image fusion, image flow, and/or any methodology affording the user to identify, define and inform various aspects of using imaging during the procedure, as displayed in the various system user interfaces and displays. Examples may include locating, displaying and characterizing regions of interest, organ systems, potential treatment sites within, with on and/or surrounding organs or tissues, identifying critical structures such as ducts, vessels, nerves, ureters, fissures, capsules, tumors, tissue trauma/injury/disease, other organs, connective tissues, etc., and/or in context to one another, of one or more (e.g., tumor draining lymphatics or vasculature; or tumor proximity to organ capsule or underlying other organ), as unlimited examples.

Systems may be configured to include onboard integrated imaging hardware, software, sensors, probes and wetware, and/or may be configured to communicate and interface with external imaging and image processing systems. The aforementioned components may be also integrated into the system's Therapy sub-system components wherein probes, imaging arrays, or the like, and electrically, mechanically or electromechanically integrated into therapy transducers. This may afford, in part, the ability to have geometrically aligned imaging and therapy, with the therapy directly within the field of view, and in some cases in line, with imaging. In some embodiments, this integration may comprise a fixed orientation of the imaging capability (e.g., imaging probe) in context to the therapy transducer. In other embodiments, the imaging solution may be able to move or adjust its position, including modifying angle, extension (e.g., distance from therapy transducer or patient), rotation (e.g., imaging plane in example of an ultrasound probe) and/or other parameters, including moving/adjusting dynamically while actively imaging. The imaging component or probe may be encoded so its orientation and position relative to another aspect of the system, such as the therapy transducer, and/or robotically-enabled positioning component may be determined.

In one embodiment, the system may comprise onboard ultrasound, further configured to allow users to visualize, monitor and receive feedback for procedure sites through the system displays and software, including allowing ultrasound imaging and characterization (and various forms of), ultrasound guided planning and ultrasound guided treatment, all in real-time. The system may be configured to allow users to manually, semi-automated or in fully automated means image the patient (e.g., by hand or using a robotically-enabled imager).

In some embodiments, imaging feedback and monitoring can include monitoring changes in: backscatter from bubble clouds; speckle reduction in backscatter; backscatter speckle statistics; mechanical properties of tissue (i.e., elastography); tissue perfusion (i.e., ultrasound contrast); shear wave propagation; acoustic emissions, electrical impedance tomography, and/or various combinations of, including as displayed or integrated with other forms of imaging (e.g., CT or MRI).

In some embodiments, imaging including feedback and monitoring from backscatter from bubble clouds, may be used as a method to determine immediately if the histotripsy process has been initiated, is being properly maintained, or even if it has been extinguished. For example, this method enables continuously monitored in real time drug delivery, tissue erosion, and the like. The method also can provide feedback permitting the histotripsy process to be initiated at a higher intensity and maintained at a much lower intensity. For example, backscatter feedback can be monitored by any transducer or ultrasonic imager. By measuring feedback for the therapy transducer, an accessory transducer can send out interrogation pulses or be configured to passively detect cavitation.

Moreover, the nature of the feedback received can be used to adjust acoustic parameters (and associated system parameters) to optimize the drug delivery and/or tissue erosion process.

In some embodiments, imaging including feedback and monitoring from backscatter, and speckle reduction, may be configured in the system.

For systems comprising feedback and monitoring via backscattering, and as means of background, as tissue is progressively mechanically subdivided, in other words homogenized, disrupted, or eroded tissue, this process results in changes in the size and distribution of acoustic scatter. At some point in the process, the scattering particle size and density is reduced to levels where little ultrasound is scattered, or the amount scattered is reduced significantly. This results in a significant reduction in speckle, which is the coherent constructive and destructive interference patterns of light and dark spots seen on images when coherent sources of illumination are used; in this case, ultrasound. After some treatment time, the speckle reduction results in a dark area in the therapy volume. Since the amount of speckle reduction is related to the amount of tissue subdivision, it can be related to the size of the remaining tissue fragments. When this size is reduced to sub-cellular levels, no cells are assumed to have survived. So, treatment can proceed until a desired speckle reduction level has been reached. Speckle is easily seen and evaluated on standard ultrasound imaging systems. Specialized transducers and systems, including those disclosed herein, may also be used to evaluate the backscatter changes.

Further, systems comprising feedback and monitoring via speckle, and as means of background, an image may persist from frame to frame and change very little as long as the scatter distribution does not change and there is no movement of the imaged object. However, long before the scatters are reduced enough in size to cause speckle reduction, they may be changed sufficiently to be detected by signal processing and other means. This family of techniques can operate as detectors of speckle statistics changes. For example, the size and position of one or more speckles in an image will begin to decorrelate before observable speckle reduction occurs. Speckle decorrelation, after appropriate motion compensation, can be a sensitive measure of the mechanical disruption of the tissues, and thus a measure of therapeutic efficacy. This feedback and monitoring technique may permit early observation of changes resulting from the acoustic cavitation/histotripsy process and can identify changes in tissue before substantial or complete tissue effect (e.g., erosion occurs). In one embodiment, this method may be used to monitor the acoustic cavitation/histotripsy process for enhanced drug delivery where treatment sites/tissue is temporally disrupted, and tissue damage/erosion is not desired. In other embodiments, this may comprise speckle decorrelation by movement of scatters in an increasingly fluidized therapy volume. For example, in the case where partial or complete tissue erosion is desired.

For systems comprising feedback and monitoring via elastography, and as means of background, as treatment sites/tissue are further subdivided per an acoustic cavitation/histotripsy effect (homogenized, disrupted, or eroded), its mechanical properties change from a soft but interconnected solid to a viscous fluid or paste with few long-range interactions. These changes in mechanical properties can be measured by various imaging modalities including MRI and ultrasound imaging systems. For example, an ultrasound pulse can be used to produce a force (i.e., a radiation force) on a localized volume of tissue. The tissue response (displacements, strains, and velocities) can change significantly during histotripsy treatment allowing the state of tissue disruption to be determined by imaging or other quantitative means.

Systems may also comprise feedback and monitoring via shear wave propagation changes. As means of background, the subdivision of tissues makes the tissue more fluid and less solid and fluid systems generally do not propagate shear waves. Thus, the extent of tissue fluidization provides opportunities for feedback and monitoring of the histotripsy process. For example, ultrasound and MRI imaging systems can be used to observe the propagation of shear waves. The extinction of such waves in a treated volume is used as a measure of tissue destruction or disruption. In one system embodiment, the system and supporting sub-systems may be used to generate and measure the interacting shear waves. For example, two adjacent ultrasound foci might perturb tissue by pushing it in certain ways. If adjacent foci are in a fluid, no shear waves propagate to interact with each other. If the tissue is not fluidized, the interaction would be detected with external means, for example, by a difference frequency only detected when two shear waves interact nonlinearly, with their disappearance correlated to tissue damage. As such, the system may be configured to use this modality to enhance feedback and monitoring of the acoustic cavitation/histotripsy procedure.

For systems comprising feedback and monitoring via acoustic emission, and as means of background, as a tissue volume is subdivided, its effect on acoustic cavitation/histotripsy (e.g., the bubble cloud here) is changed. For example, bubbles may grow larger and have a different lifetime and collapse changing characteristics in intact versus fluidized tissue. Bubbles may also move and interact after tissue is subdivided producing larger bubbles or cooperative interaction among bubbles, all of which can result in changes in acoustic emission. These emissions can be heard during treatment and they change during treatment. Analysis of these changes, and their correlation to therapeutic efficacy, enables monitoring of the progress of therapy, and may be configured as a feature of the system.

For systems comprising feedback and monitoring via electrical impedance tomography, and as means of background, an impedance map of a therapy site can be produced based upon the spatial electrical characteristics throughout the therapy site. Imaging of the conductivity or permittivity of the therapy site of a patient can be inferred from taking skin surface electrical measurements. Conducting electrodes are attached to a patient's skin and small alternating currents are applied to some or all of the electrodes. One or more known currents are injected into the surface and the voltage is measured at a number of points using the electrodes. The process can be repeated for different configurations of applied current. The resolution of the resultant image can be adjusted by changing the number of electrodes employed. A measure of the electrical properties of the therapy site within the skin surface can be obtained from the impedance map, and changes in and location of the acoustic cavitation/histotripsy (e.g., bubble cloud, specifically) and histotripsy process can be monitored using this as configured in the system and supporting sub-systems.

The user may be allowed to further select, annotate, mark, highlight, and/or contour, various regions of interest or treatment sites, and defined treatment targets (on the image(s)), of which may be used to command and direct the system where to image, test and/or treat, through the system software and user interfaces and displays. In some arrangements, the user may use a manual ultrasound probe (e.g., diagnostic hand-held probe) to conduct the procedure. In another arrangement, the system may use a robot and/or electromechanical positioning system to conduct the procedure, as directed and/or automated by the system, or conversely, the system can enable combinations of manual and automated uses.

The system may further include the ability to conduct image registration, including imaging and image data set registration to allow navigation and localization of the system to the patient, including the treatment site (e.g., tumor, critical structure, bony anatomy, anatomy and identifying features of, etc.). In one embodiment, the system allows the user to image and identify a region of interest, for example the liver, using integrated ultrasound, and to select and mark a tumor (or surrogate marker of) comprised within the liver through/displayed in the system software, and wherein said system registers the image data to a coordinate system defined by the system, that further allows the system's Therapy and Robotics sub-systems to deliver synchronized acoustic cavitation/histotripsy to said marked tumor. The system may comprise the ability to register various image sets, including those previously disclosed, to one another, as well as to afford navigation and localization (e.g., of a therapy transducer to a CT or MRI/ultrasound fusion image with the therapy transducer and Robotics sub-system tracking to said image).

The system may also comprise the ability to work in a variety of interventional, endoscopic and surgical environments, including alone and with other systems (surgical/laparoscopic towers, vision systems, endoscope systems and towers, ultrasound enabled endoscopic ultrasound (flexible and rigid), percutaneous/endoscopic/laparoscopic and minimally invasive navigation systems (e.g., optical, electromagnetic, shape-sensing, ultrasound-enabled, etc.), of also which may work with, or comprise various optical imaging capabilities (e.g., fiber and or digital). The disclosed system may be configured to work with these systems, in some embodiments working alongside them in concert, or in other embodiments where all or some of the system may be integrated into the above systems/platforms (e.g., acoustic cavitation/histotripsy-enabled endoscope system or laparoscopic surgical robot). In many of these environments, a therapy transducer may be utilized at or around the time of use, for example, of an optically guided endoscope/bronchoscope, or as another example, at the time a laparoscopic robot (e.g., Intuitive Da Vinci*Xi system) is viewing/manipulating a tissue/treatment site. Further, these embodiments and examples may include where said other systems/platforms are used to deliver (locally) fluid to enable the creation of a man-made acoustic window, where on under normal circumstances may not exist (e.g., fluidizing a segment or lobe of the lung in preparation for acoustic cavitation/histotripsy via non-invasive transthoracic treatment (e.g., transducer externally placed on/around patient). Systems disclosed herein may also comprise all or some of their sub-system hardware packaged within the other system cart/console/systems described here (e.g., acoustic cavitation/histotripsy system and/or sub-systems integrated and operated from said navigation or laparoscopic system).

The system may also be configured, through various aforementioned parameters and other parameters, to display real-time visualization of a bubble cloud in a spatial-temporal manner, including the resulting tissue effect peri/post-treatment from tissue/bubble cloud interaction, wherein the system can dynamically image and visualize, and display, the bubble cloud, and any changes to it (e.g., decreasing or increasing echogenicity), which may include intensity, shape, size, location, morphology, persistence, etc. These features may allow users to continuously track and follow the treatment in real-time in one integrated procedure and interface/system, and confirm treatment safety and efficacy on the fly (versus other interventional or surgical modalities, which either require multiple procedures to achieve the same, or where the treatment effect is not visible in real-time (e.g., radiation therapy), or where it is not possible to achieve such (e.g., real-time visualization of local tissue during thermal ablation), and/or where the other procedure further require invasive approaches (e.g., incisions or punctures) and iterative imaging in a scanner between procedure steps (e.g., CT or MRI scanning). The above disclosed systems, sub-systems, components, modalities, features and work-flows/methods of use may be implemented in an unlimited fashion through enabling hardware, software, user interfaces and use environments, and future improvements, enhancements and inventions in this area are considered as included in the scope of this disclosure, as well as any of the resulting data and means of using said data for analytics, artificial intelligence or digital health applications and systems.

### Robotics

They system may comprise various Robotic sub-systems and components, including but not limited to, one or more robotic arms and controllers, which may further work with other sub-systems or components of the system to deliver and monitor acoustic cavitation/histotripsy. As previously discussed herein, robotic arms and control systems may be integrated into one or more Cart configurations.

For example, one system embodiment may comprise a Cart with an integrated robotic arm and control system, and Therapy, Integrated Imaging and Software, where the robotic arm and other listed sub-systems are controlled by the user through the form factor of a single bedside Cart.

In other embodiments, the Robotic sub-system may be configured in one or more separate Carts, that may be a driven in a master/slave configuration from a separate master or Cart, wherein the robotically-enabled Cart is positioned bed/patient-side, and the Master is at a distance from said Cart.

Disclosed robotic arms may be comprised of a plurality of joints, segments, and degrees of freedom and may also include various integrated sensor types and encoders, implemented for various use and safety features. Sensing technologies and data may comprise, as an example, vision, potentiometers, position/localization, kinematics, force, torque, speed, acceleration, dynamic loading, and/or others. In some cases, sensors may be used for users to direct robot commands (e.g., hand gesture the robot into a preferred set up position, or to dock home). Additional details on robotic arms can be found in U.S. Patent Pub. No. 2013/0255426 A1 to Kassow et al., which is disclosed herein by reference in its entirety.

The robotic arm receives control signals and commands from the robotic control system, which may be housed in a Cart. The system may be configured to provide various functionalities, including but not limited to, position, tracking, patterns, triggering, and events/actions.

Position may be configured to comprise fixed positions, pallet positions, time-controlled positions, distance-controlled positions, variable-time controlled positions, variable-distance controlled positions.

Tracking may be configured to comprise time-controlled tracking and/or distance-controlled tracking.

The patterns of movement may be configured to comprise intermediate positions or waypoints, as well as sequence of positions, through a defined path in space.

Triggers may be configured to comprise distance measuring means, time, and/or various sensor means including those disclosed herein, and not limited to, visual/imaging-based, force, torque, localization, energy/power feedback and/or others.

Events/actions may be configured to comprise various examples, including proximity-based (approaching/departing a target object), activation or de-activation of various end-effectors (e.g., therapy transducers), starting/stopping/pausing sequences of said events, triggering or switching between triggers of events/actions, initiating patterns of movement and changing/toggling between patterns of movement, and/or time-based and temporal over the defined work and time-space.

In one embodiment, the system comprises a three degree of freedom robotic positioning system, enabled to allow the user (through the software of the system and related user interfaces), to micro-position a therapy transducer through X, Y, and Z coordinate system, and where gross macro-positioning of the transducer (e.g., aligning the transducer on the patient's body) is completed manually. In some embodiments, the robot may comprise 6 degrees of freedom including X, Y, Z, and pitch, roll and yaw. In other embodiments, the Robotic sub-system may comprise further degrees of freedom, that allow the robot arm supporting base to be positioned along a linear axis running parallel to the general direction of the patient surface, and/or the supporting base height to be adjusted up or down, allowing the position of the robotic arm to be modified relative to the patient, patient surface, Cart, Coupling sub-system, additional robots/robotic arms and/or additional surgical systems, including but not limited to, surgical towers, imaging systems, endoscopic/laparoscopic systems, and/or other.

One or more robotic arms may also comprise various features to assist in maneuvering and modifying the arm position, manually or semi-manually, and of which said features may interface on or between the therapy transducer and the most distal joint of the robotic arm. In some embodiments, the feature is configured to comprise a handle allowing maneuvering and manual control with one or more hands. The handle may also be configured to include user input and electronic control features of the robotic arm, to command various drive capabilities or modes, to actuate the robot to assist in gross or fine positioning of the arm (e.g., activating or deactivating free drive mode). The work-flow for the initial positioning of the robotic arm and therapy head can be configured to allow either first positioning the therapy transducer/head in the coupling solution, with the therapy transducer directly interfaced to the arm, or in a different work-flow, allowing the user to set up the coupling solution first, and enabling the robot arm to be interfaced to the therapy transducer/coupling solution as a later/terminal set up step.

In some embodiments, the one or more robotic arms or other features of the robotic sub-systems may include sensors or other features configured to measure, determine, or predict the force(s) acting against the robotic arm(s) and/or the therapy transducer array coupled to the robotic arm(s). These sensors can include force sensors or force transducers not limited to load cells, pneumatic load cells, capacitive load cells, strain gauge load cells, hydraulic load cells, etc. In some implementations, the force sensors can be disposed on or in the robotic arm(s), on or in the transducer array or therapy probe, on or in the coupling linkages between the transducer array and robotic arm, or in any other location within the system, including the robotics sub-system, where a force sensor or sensors would be adapted and configured to measure the force applied against the robotic arm or the transducer array. Additionally, these force sensors can be electronically or operatively coupled to any of the control systems described herein, including electronic controllers, robotic positioning systems, navigation systems, or any other cpus, processors, or controllers configured to control the operation of the transducer array, robotics sub-system, or any other sub-system during therapy.

In some embodiments, the robotic arm may comprise a robotic arm on a laparoscopic, single port, endoscopic, hybrid or combination of, and/or other robot, wherein said robot of the system may be a slave to a master that controls said arm, as well as potentially a plurality of other arms, equipped to concurrently execute other tasks (vision, imaging, grasping, cutting, ligating, sealing, closing, stapling, ablating, suturing, marking, etc.), including actuating one or more laparoscopic arms (and instruments) and various histotripsy system components. For example, a laparoscopic robot may be utilized to prepare the surgical site, including manipulating organ position to provide more ideal acoustic access and further stabilizing said organ in some cases to minimize respiratory motion. In conjunction and parallel to this, a second robotic arm may be used to deliver non-invasive acoustic cavitation through a body cavity, as observed under real-time imaging from the therapy transducer (e.g., ultrasound) and with concurrent visualization via a laparoscopic camera. In other related aspects, a similar approach may be utilized with a combination of an endoscopic and non-invasive approach, and further, with a combination of an endoscopic, laparoscopic and non-invasive approach.

### Software

The system may comprise various software applications, features and components which allow the user to interact, control and use the system for a plethora of clinical applications. The Software may communicate and work with one or more of the sub-systems, including but not limited to Therapy, Integrated Imaging, Robotics and Other Components, Ancillaries and Accessories of the system.

Overall, in no specific order of importance, the software may provide features and support to initialize and set up the system, service the system, communicate and import/export/store data, modify/manipulate/configure/control/command various settings and parameters by the user, mitigate safety and use-related risks, plan procedures, provide support to various configurations of transducers, robotic arms and drive systems, function generators and amplifier circuits/slaves, test and treatment ultrasound sequences, transducer steering and positioning (electromechanical and electronic beam steering, etc.), treatment patterns, support for imaging and imaging probes, manual and electromechanical/robotically-enabling movement of, imaging support for measuring/characterizing various dimensions within or around procedure and treatment sites (e.g., depth from one anatomical location to another, etc., pre-treatment assessments and protocols for measuring/characterizing in situ treatment site properties and conditions (e.g., acoustic cavitation/histotripsy thresholds and heterogeneity of), targeting and target alignment, calibration, marking/annotating, localizing/navigating, registering, guiding, providing and guiding through work-flows, procedure steps, executing treatment plans and protocols autonomously, autonomously and while under direct observation and viewing with real-time imaging as displayed through the software, including various views and viewports for viewing, communication tools (video, audio, sharing, etc.), troubleshooting, providing directions, warnings, alerts, and/or allowing communication through various networking devices and protocols. It is further envisioned that the software user interfaces and supporting displays may comprise various buttons, commands, icons, graphics, text, etc., that allow the user to interact with the system in a user-friendly and effective manner, and these may be presented in an unlimited number of permutations, layouts and designs, and displayed in similar or different manners or feature sets for systems that may comprise more than one display (e.g., touch screen monitor and touch pad), and/or may network to one or more external displays or systems (e.g., another robot, navigation system, system tower, console, monitor, touch display, mobile device, tablet, etc.).

The software, as a part of a representative system, including one or more computer processors, may support the various aforementioned function generators (e.g., FPGA), amplifiers, power supplies and therapy transducers. The software may be configured to allow users to select, determine and monitor various parameters and settings for acoustic cavitation/histotripsy, and upon observing/receiving feedback on performance and conditions, may allow the user to stop/start/modify said parameters and settings.

The software may be configured to allow users to select from a list or menu of multiple transducers and support the auto-detection of said transducers upon connection to the system (and verification of the appropriate sequence and parameter settings based on selected application). In other embodiments, the software may update the targeting and amplifier settings (e.g., channels) based on the specific transducer selection. The software may also provide transducer recommendations based on pre-treatment and planning inputs. Conversely, the software may provide error messages or warnings to the user if said therapy transducer, amplifier and/or function generator selections or parameters are erroneous, yield a fault or failure. This may further comprise reporting the details and location of such.

In addition to above, the software may be configured to allow users to select treatment sequences and protocols from a list or menu, and to store selected and/or previous selected sequences and protocols as associated with specific clinical uses or patient profiles. Related profiles may comprise any associated patient, procedure, clinical and/or engineering data, and maybe used to inform, modify and/or guide current or future treatments or procedures/interventions, whether as decision support or an active part of a procedure itself (e.g., using serial data sets to build and guide new treatments).

As a part of planning or during the treatment, the software (and in working with other components of the system) may allow the user to evaluate and test acoustic cavitation/histotripsy thresholds at various locations in a user-selected region of interest or defined treatment area/volume, to determine the minimum cavitation thresholds throughout said region or area/volume, to ensure treatment parameters are optimized to achieve, maintain and dynamically control acoustic cavitation/histotripsy. In one embodiment, the system allows a user to manually evaluate and test threshold parameters at various points. Said points may include those at defined boundary, interior to the boundary and center locations/positions, of the selected region of interest and treatment area/volume, and where resulting threshold measurements may be reported/displayed to the user, as well as utilized to update therapy parameters before treatment. In another embodiment, the system may be configured to allow automated threshold measurements and updates, as enabled by the aforementioned Robotics sub-system, wherein the user may direct the robot, or the robot may be commanded to execute the measurements autonomously.

Software may also be configured, by working with computer processors and one or more function generators, amplifiers and therapy transducers, to allow various permutations of delivering and positioning optimized acoustic cavitation/histotripsy in and through a selected area/volume. This may include, but not limited to, systems configured with a fixed/natural focus arrangement using purely electromechanical positioning configuration(s), electronic beam steering (with or without electromechanical positioning), electronic beam steering to a new selected fixed focus with further electromechanical positioning, axial (Z axis) electronic beam steering with lateral (X and Y) electromechanical positioning, high speed axial electronic beam steering with lateral electromechanical positioning, high speed beam steering in 3D space, various combinations of including with dynamically varying one or more acoustic cavitation/histotripsy parameters based on the aforementioned ability to update treatment parameters based on threshold measurements (e.g., dynamically adjusting amplitude across the treatment area/volume).

### Other Components, Ancillaries and Accessories

The system may comprise various other components, ancillaries and accessories, including but not limited to computers, computer processors, power supplies including high voltage power supplies, controllers, cables, connectors, networking devices, software applications for security, communication, integration into information systems including hospital information systems, cellular communication devices and modems, handheld wired or wireless controllers, goggles or glasses for advanced visualization, augmented or virtual reality applications, cameras, sensors, tablets, smart devices, phones, internet of things enabling capabilities, specialized use “apps” or user training materials and applications (software or paper based), virtual proctors or trainers and/or other enabling features, devices, systems or applications, and/or methods of using the above.

### System Variations and Methods/Applications

In addition to performing a breadth of procedures, the system may allow additional benefits, such as enhanced planning, imaging and guidance to assist the user. In one embodiment, the system may allow a user to create a patient, target and application specific treatment plan, wherein the system may be configured to optimize treatment parameters based on feedback to the system during planning, and where planning may further comprise the ability to run various test protocols to gather specific inputs to the system and plan.

Feedback may include various energy, power, location, position, tissue and/or other parameters.

The system, and the above feedback, may also be further configured and used to autonomously (and robotically) execute the delivery of the optimized treatment plan and protocol, as visualized under real-time imaging during the procedure, allowing the user to directly observe the local treatment tissue effect, as it progresses through treatment, and start/stop/modify treatment at their discretion. Both test and treatment protocols may be updated over the course of the procedure at the direction of the user, or in some embodiments, based on logic embedded within the system.

It is also recognized that many of these benefits may further improve other forms of acoustic therapy, including thermal ablation with high intensity focused ultrasound (HIFU), high intensity therapeutic ultrasound (HITU) including boiling histotripsy (thermal cavitation), and are considered as part of this disclosure. The disclosure also considers the application of histotripsy as a means to activate previously delivered in active drug payloads whose activity is inert due to protection in a micelle, nanostructure or similar protective structure or through molecular arrangement that allows activation only when struck with acoustic energy.

In another aspect, the Therapy sub-system, comprising in part, one or more amplifiers, transducers and power supplies, may be configured to allow multiple acoustic cavitation and histotripsy driving capabilities, affording specific benefits based on application, method and/or patient specific use. These benefits may include, but are not limited to, the ability to better optimize and control treatment parameters, which may allow delivery of more energy, with more desirable thermal profiles, increased treatment speed and reduced procedure times, enable electronic beam steering and/or other features.

This disclosure also includes novel systems and concepts as related to systems and sub-systems comprising new and “universal” amplifiers, which may allow multiple driving approaches (e.g., single and multi-cycle pulsing). In some embodiments, this may include various novel features to further protect the system and user, in terms of electrical safety or other hazards (e.g., damage to transducer and/or amplifier circuitry).

In another aspect, the system, and Therapy sub-system, may include a plethora of therapy transducers, where said therapy transducers are configured for specific applications and uses and may accommodate treating over a wide range of working parameters (target size, depth, location, etc.) and may comprise a wide range of working specifications (detailed below). Transducers may further adapt, interface and connect to a robotically-enabled system, as well as the Coupling sub-system, allowing the transducer to be positioned within, or along with, an acoustic coupling device allowing, in many embodiments, concurrent imaging and histotripsy treatments through an acceptable acoustic window. The therapy transducer may also comprise an integrated imaging probe or localization sensors, capable of displaying and determining transducer position within the treatment site and affording a direct field of view (or representation of) the treatment site, and as the acoustic cavitation/histotripsy tissue effect and bubble cloud may or may not change in appearance and intensity, throughout the treatment, and as a function of its location within said treatment (e.g., tumor, healthy tissue surrounding, critical structures, adipose tissue, etc.).

The systems, methods and use of the system disclosed herein, may be beneficial to overcoming significant unmet needs in the areas of soft tissue ablation, oncology, immuno-oncology, advanced image guided procedures, surgical procedures including but not limited to open, laparoscopic, single incision, natural orifice, endoscopic, non-invasive, various combination of, various interventional spaces for catheter-based procedures of the vascular, cardiovascular pulmonary and/or neurocranial-related spaces, cosmetics/aesthetics, metabolic (e.g., type 2 diabetes), plastic and reconstructive, ocular and ophthalmology, orthopedic, gynecology and men's health, and other systems, devices and methods of treating diseased, injured, undesired, or healthy tissues, organs or cells.

Systems and methods are also provided for improving treatment patterns within tissue that can reduce treatment time, improve efficacy, and reduce the amount of energy and prefocal tissue heating delivered to patients.

### Use Environments

The disclosed system, methods of use, and use of the system, may be conducted in a plethora of environments and settings, with or without various support systems such as anesthesia, including but not limited to, procedure suites, operating rooms, hybrid rooms, in and out-patient settings, ambulatory settings, imaging centers, radiology, radiation therapy, oncology, surgical and/or any medical center, as well as physician offices, mobile healthcare centers or systems, automobiles and related vehicles (e.g., van), aero and marine transportation vehicles such as planes and ships, and/or any structure capable of providing temporary procedure support (e.g., tent). In some cases, systems and/or sub-systems disclosed herein may also be provided as integrated features into other environments, for example, the direct integration of the histotripsy Therapy sub-system into a MRI scanner or patient surface/bed, wherein at a minimum the therapy generator and transducer are integral to such, and in other cases wherein the histotripsy configuration further includes a robotic positioning system, which also may be integral to a scanner or bed centered design.

### Coupling

Systems may comprise a variety of Coupling sub-system embodiments, of which are enabled and configured to allow acoustic coupling to the patient to afford effective acoustic access for ultrasound visualization and acoustic cavitation/histotripsy (e.g., provide acoustic window and medium between the transducer(s) and patient, and support of). These may include different form factors of such, including open and enclosed device solutions, and some arrangements which may be configured to allow dynamic control over the acoustic medium (e.g., temperature, dissolved gas content, level of particulate filtration, sterility, volume, composition, etc.). Such dynamic control components may be directly integrated to the system (within the Cart), or may be in temporary/intermittent or continuous communication with the system, but externally situated in a separate device and/or cart.

The Coupling sub-system typically comprises, at a minimum, coupling medium (e.g., degassed water or water solutions), a reservoir/container to contain said coupling medium, and a support structure (including interfaces to other surfaces or devices). In most embodiments, the coupling medium is water, and wherein the water may be conditioned before or during the procedure (e.g., chilled, degassed, filtered, etc.). Various conditioning parameters may be employed based on the configuration of the system and its intended use/application.

The reservoir or medium container may be formed and shaped to various sizes and shapes, and to adapt/conform to the patient, allow the therapy transducer to engage/access and work within the acoustic medium, per defined and required working space (minimum volume of medium to allow the therapy transducer to be positioned and/or move through one or more treatment positions or patterns, and at various standoffs or depths from the patient, etc.), and wherein said reservoir or medium container may also mechanically support the load, and distribution of the load, through the use of a mechanical and/or electromechanical support structure. As a representative example, this may include a support frame. The container may be of various shapes, sizes, curvatures, and dimensions, and may be comprised of a variety of materials compositions (single, multiple, composites, etc.), of which may vary throughout. In some embodiments, it may comprise features such as films, drapes, membranes, bellows, etc. that may be insertable and removable, and/or fabricated within, of which may be used to conform to the patient and assist in confining/containing the medium within the container. It may further contain various sensors (e.g., volume/fill level), drains (e.g., inlet/outlet), lighting (e.g., LEDs), markings (e.g., fill lines, set up orientations, etc.), text (e.g., labeling), etc.

In one embodiment, the reservoir or medium container contains a sealable frame, of which a membrane and/or film may be positioned within, to afford a conformable means of contacting the reservoir (later comprising the treatment head/therapy transducer) as an interface to the patient, that further provides a barrier to the medium (e.g., water) between the patient and therapy transducer). In other embodiments, the membrane and/or film may comprise an opening, the patient contacting edge of which affords a fluid/mechanical seal to the patient, but in contrast allows medium communication directly with the patient (e.g., direct degassed water interface with patient). The superstructure of the reservoir or medium container in both these examples may further afford the proximal portion of the structure (e.g., top) to be open or enclosed (e.g., to prevent spillage or afford additional features).

Disclosed membranes may be comprised of various elastomers, viscoelastic polymers, thermoplastics, thermoplastic elastomers, thermoset polymers, silicones, urethanes, rigid/flexible co-polymers, block co-polymers, random block co-polymers, etc. Materials may be hydrophilic, hydrophobic, surface modified, coated, extracted, etc., and may also contain various additives to enhance performance, appearance or stability. In some embodiments, the thermoplastic elastomer may be styrene-ethylene-butylene-styrene (SEBS), or other like strong and flexible elastomers. The membrane form factor can be flat or pre-shaped prior to use. In other embodiments, the membrane could be inelastic (i.e., a convex shape) and pressed against the patient's skin to acoustically couple the transducer to the tissue. Systems and methods are further disclosed to control the level of contaminants (e.g., particulates, etc.) on the membrane to maintain the proper level of ultrasound coupling. Too many particulates or contaminants can cause scattering of the ultrasound waves. This can be achieved with removable films or coatings on the outer surfaces of the membrane to protect against contamination.

Said materials may be formed into useful membranes through molding, casting, spraying, ultrasonic spraying, extruding, and/or any other processing methodology that produces useful embodiments. They may be single use or reposable/reusable. They may be provided non-sterile, aseptically cleaned or sterile, where sterilization may comprise any known method, including but not limited to ethylene oxide, gamma, e-beam, autoclaving, steam, peroxide, plasma, chemical, etc. Membranes can be further configured with an outer molded or over molded frame to provide mechanical stability to the membrane during handling including assembly, set up and take down of the coupling sub-system. Various parameters of the membrane can be optimized for this method of use, including thickness, thickness profile, density, formulation (e.g., polymer molecular weight and copolymer ratios, additives, plasticizers, etc.), including optimizing specifically to maximize acoustic transmission properties, including minimizing impact to cavitation initiation threshold values, and/or ultrasound imaging artifacts, including but not limited to membrane reflections, as representative examples.

Open reservoirs or medium containers may comprise various methods of filling, including using pre-prepared medium or water, that may be delivered into the containers, in some cases to a defined specification of water (level of temperature, gas saturation, etc.), or they may comprise additional features integral to the design that allow filling and draining (e.g., ports, valves, hoses, tubing, fittings, bags, pumps, etc.). These features may be further configured into or to interface to other devices, including for example, a fluidics system. In some cases, the fluidics system may be an in-house medium preparation system in a hospital or care setting room, or conversely, a mobile cart-based system which can prepare and transport medium to and from the cart to the medium container, etc.

Enclosed iterations of the reservoir or medium container may comprise various features for sealing, in some embodiments sealing to a proximal/top portion or structure of a reservoir/container, or in other cases where sealing may comprise embodiments that seal to the transducer, or a feature on the transducer housings. Further, some embodiments may comprise the dynamic ability to control the volume of fluid within these designs, to minimize the potential for air bubbles or turbulence in said fluid and to allow for changes in the focal length to the target area without moving the transducer. As such, integrated features allowing fluid communication, and control of, may be provided (ability to provide/remove fluid on demand), including the ability to monitor and control various fluid parameters, some disclosed above. In order to provide this functionality, the overall system, and as part, the Coupling sub-system, may comprise a fluid conditioning system, which may contain various electromechanical devices, systems, power, sensing, computing, pumping, filtering and control systems, etc. The reservoir may also be configured to receive signals that cause it to deform or change shape in a specific and controlled manner to allow the target point to be adjusted without moving the transducer.

Coupling support systems may include various mechanical support devices to interface the reservoir/container and medium to the patient, and the workspace (e.g., bed, floor, etc.). In some embodiments, the support system comprises a mechanical arm with 3 or more degrees of freedom. Said arm may have a proximal interface with one or more locations (and features) of the bed, including but not limited to, the frame, rails, customized rails or inserts, as well as one or more distal locations of the reservoir or container. The arm may also be a feature implemented on one or more Carts, wherein Carts may be configured in various unlimited permutations, in some cases where a Cart only comprises the role of supporting and providing the disclosed support structure.

In some embodiments, the support structure and arm may be a robotically-enabled arm, implemented as a stand-alone Cart, or integrated into a Cart further comprising two or more system sub-systems, or where in the robotically-enabled arm is an arm of another robot, of interventional, surgical or other type, and may further comprise various user input features to actuate/control the robotic arm (e.g., positioning into/within coupling medium) and/or Coupling solution features (e.g., filling, draining, etc.). In some examples, the support structure robotic arm positional encoders may be used to coordinate the manipulation of the second arm (e.g. comprising the therapy transducer/treatment head), such as to position the therapy transducer to a desired/known location and pose within the coupling support structure.

Overall, significant unmet needs exist in interventional and surgical medical procedures today, including those procedures utilizing minimally invasive devices and approaches to treat disease and/or injury, and across various types of procedures where the unmet needs may be solved with entirely new medical procedures. Today's medical system capabilities are often limited by access, wherein a less or non-invasive approach would be preferred, or wherein today's tools aren't capable to deliver preferred/required tissue effects (e.g., operate around/through critical structures without serious injury), or where the physical set up of the systems makes certain procedure approaches less desirable or not possible, and where a combination of approaches, along with enhanced tissue effecting treatments, may enable entirely new procedures and approaches, not possible today.

In addition, specific needs exist for enabling histotripsy delivery, including robotic histotripsy delivery, wherein one or more histotripsy therapy transducers may be configured to acoustically couple to a patient, using a completely sealed approach (e.g., no acoustic medium communication with the patient's skin) and allowing the one or more histotripsy transducers to be moved within the coupling solution without impeding the motion/movement of the robotic arm or interfering/disturbing the coupling interface, which could affect the intended treatment and/or target location.

Disclosed herein are histotripsy acoustic and patient coupling systems and methods, to enable histotripsy therapy/treatment, as envisioned in any setting, from interventional suite, operating room, hybrid suites, imaging centers, medical centers, office settings, mobile treatment centers, and/or others, as non-limiting examples. The following disclosure further describes novel systems used to create, control, maintain, modify/enhance, monitor and setup/takedown acoustic and patient coupling systems, in a variety of approaches, methods, environments, architectures and work-flows. In general, the disclosed novel systems may allow for a coupling medium, in some examples degassed water, to be interfaced between a histotripsy therapy transducer and a patient, wherein the acoustic medium provides sufficient acoustic coupling to said patient, allowing the delivery of histotripsy pulses through a user desired treatment location (and volume), where the delivery may require physically moving the histotripsy therapy transducer within a defined work-space comprising the coupling medium, and also where the coupling system is configured to allow said movement of the therapy transducer (and positioning system, e.g., robot) freely and unencumbered from by the coupling support system (e.g., a frame or manifold holding the coupling medium).

### Coupling System and Sub-Systems/Components

The disclosed histotripsy acoustic and patient coupling systems, in general, may comprise one or more of the following sub-systems and components, including but not limited to 1) a membrane/barrier film to provide an enclosed, sealed and conformal patient coupling and histotripsy system interface, 2) a frame and assembly to retain the membrane and provide sufficient work and head space for a histotripsy therapy transducers required range of motion (x, y and z, pitch, roll and yaw), 3) a sufficient volume of ultrasound medium to afford acoustic coupling and interfaces to a histotripsy therapy transducer and robotic arm, 4) one or more mechanical support arms to allow placement, positioning and load support of the frame, assembly and medium and 5) a fluidics system to prepare, provide and remove ultrasound medium(s) from the frame and assembly.

In some embodiments, the coupling system may be fully sealed, and in other embodiments and configurations, it may be partially open to afford immediate access (physical and/or visual).

The acoustic and patient coupling systems and sub-systems may further comprise various features and functionality, and associated work-flows, and may also be configured in a variety of ways to enable histotripsy procedures as detailed below.

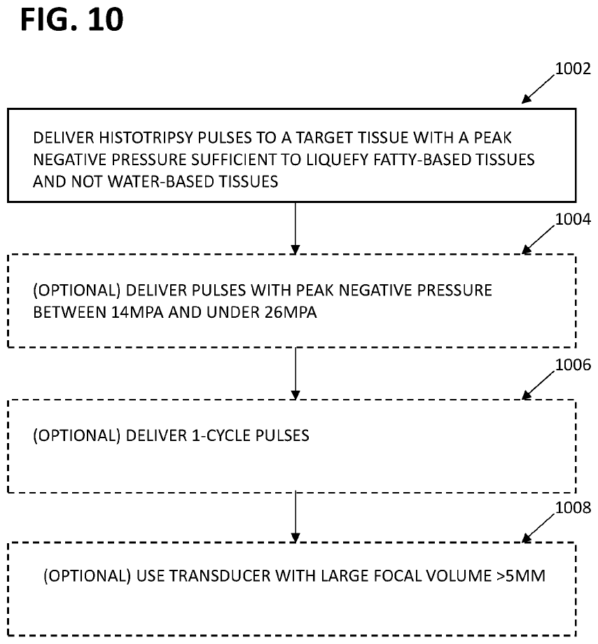

The disclosed histotripsy acoustic and patient coupling systems, in general, may comprise one or more of the following sub-systems and components, an example of which is depicted in at least FIG. 10, including but not limited to 1) a membrane/barrier film to provide an enclosed, sealed and conformal patient coupling and histotripsy system interface, 2) a frame and assembly to retain the membrane and provide sufficient work and head space for a histotripsy therapy transducers required range of motion (x, y and z, pitch, roll and yaw), 3) a sufficient volume of ultrasound medium to afford acoustic coupling and interfaces to a histotripsy therapy transducer and robotic arm, 4) one or more mechanical support arms to allow placement, positioning and load support of the frame, assembly and medium and 5) a fluidics system to prepare, provide and remove ultrasound medium(s) from the frame and assembly.

In some embodiments, the coupling system may be fully sealed, and in other embodiments and configurations, it may be partially open to afford immediate access (physical and/or visual).

The acoustic and patient coupling systems and sub-systems may further comprise various features and functionality, and associated work-flows, and may also be configured in a variety of ways to enable histotripsy procedures as detailed below.

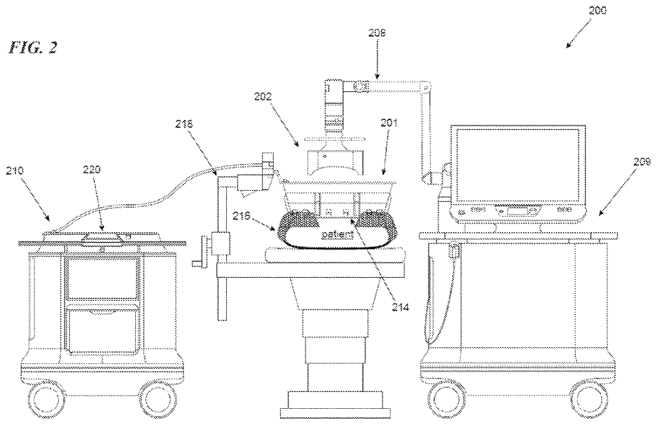

FIG. 2 illustrates one embodiment of a histotripsy therapy and imaging system 200, including a coupling assembly 201. As described above, a histotripsy therapy and imaging system can include a therapy transducer 202, an imaging system, a robotic positioning arm 208, and a fluidics cart 210. The robotic positioning arm may be attached to a therapy cart, such as cart 209.

The therapy and/or imaging transducers can be disposed within the coupling assembly 201 which can further include a coupling membrane 214 and a membrane constraint 216 configured to prevent the membrane from expanding too far from the transducer. The coupling membrane can be filled with an acoustic coupling medium such as a fluid or a gel. The membrane constraint can be, for example, a semi-rigid or rigid material as compared to the membrane, and configured to restrict expansion/movement of the membrane. In some embodiments, the membrane constraint is not used, and the elasticity and tensile strength of the membrane prevent over expansion. The coupling membrane can be a mineral-oil infused SEBS membrane to prevent direct fluid contact with the patient's skin. In the illustrated embodiment, the coupling assembly 201 is supported by a mechanical support arm 218 which can be load bearing in the x-y plane but allow for manual or automated z-axis adjustment. The mechanical support arm can be attached to the floor, the patient table, or the fluidics cart 210. The mechanical support is designed and configured to conform and hold the coupling membrane 214 in place against the patient's skin while still allowing movement of the therapy/imaging transducer relative to the patient and also relative to the coupling membrane 214 with the robotic positioning arm 208.

The fluidics cart 210 can include additional features, including a fluid tank 220, a cooling and degassing system, and a programmable control system. The fluidics cart is configured for external loading of the coupling membrane with automated control of fluidic sequences. Further details on the fluidics cart are provided below.

The therapy and/or imaging transducers can be housed in a coupling assembly which can further include a coupling membrane and a membrane constraint configured to prevent the membrane from expanding too far from the transducer. The coupling membrane can be filled with an acoustic coupling medium such as a fluid or a gel. The membrane constraint can be, for example, a semi-rigid or rigid material configured to restrict expansion/movement of the membrane. In some embodiments, the membrane constraint is not used, and the elasticity and tensile strength of the membrane prevent over expansion. The coupling membrane can be a mineral-oil infused SEBS membrane to prevent direct fluid contact with the patient's skin. In the illustrated embodiment, the coupling assembly is supported by a mechanical support arm which can be load bearing in the x-y plane but allow for manual or automated z-axis adjustment. The mechanical support arm can be attached to the floor, the patient table, or the cart. The mechanical support is designed and configured to conform and hold the coupling membrane in place against the patient's skin while still allowing movement of the therapy/imaging transducer relative to the patient and also relative to the coupling membrane with the robotic positioning arm.

The system can further include a fluidics system that can include a fluid source, a cooling and degassing system, and a programmable control system. The fluidics system is configured for external loading of the coupling membrane with automated control of fluidic sequences. Further details on the fluidics system are provided below.

### Membranes/Barrier Films and Related Architectures

Membranes and barrier films may be composed of various biocompatible materials which allow conformal coupling to patient anatomy with minimal or no entrapped bubbles capable of interfering with ultrasound imaging and histotripsy therapy, and that are capable of providing a sealed barrier layer between said patient anatomy and the ultrasound medium, of which is contained within the work-space provided by the frame and assembly.

Membrane and barrier film materials may comprise flexible and elastomeric biocompatible materials/polymers, such as various thermoplastic and thermoset materials, as well as permanent or bioresorbable polymers. Additionally, the frame of the UMC can also comprise the same materials. In some examples, the membrane may be rigid or semi-rigid polymers which are pre-shaped or flat.

### Ultrasound Medium

As previously described, the ultrasound medium may comprise any applicable medium capable of providing sufficient and useful acoustic coupling to allow histotripsy treatments and enable sufficient clinical imaging (e.g., ultrasound). Ultrasound mediums, as a part of this disclosure and system, may comprise, but are not limited to, various aqueous solutions/mediums, including mixtures with other co-soluble fluids, of which may have preferred or more preferred acoustic qualities, including ability to match speed of sound, etc. Example mediums may comprise degassed water and/or mixtures/co-solutions of degassed water and various alcohols, such as ethanol.

However, because the speed of sound in water is lower than that in tissue, sound waves can experience aberration as they travel along the water-tissue path. The source of this aberration is two-fold. Firstly, when sound waves move from a medium of lower sound speed to a medium of higher sound speed, the angle of transmission will be greater than the angle of incidence—i.e., they will bend away from the normal to the surface (Snell's Law). Secondly, the paths traversed by sound waves originating from different regions of the transducer aperture will be composed of different relative distances through each medium—i.e., the ratio of the coupling medium path length to the tissue path length is variable across the transducer. As a result of these effects, sound waves will arrive at the geometric focus of the transducer out of phase and the sound amplitude generated via constructive interference at the intended target will be reduced. Furthermore, these phase aberrations can cause a spatial shift in the point of maximal constructive interference in the sound field (i.e. a focal shift), resulting in therapeutic effect occurring at an unanticipated location.

This disclosure provides coupling mediums with advantages compared to the existing solutions. When the sound speed of the coupling medium is designed to match that of the overlaying tissue path these aberration effects are minimized. Using a Medium for Enhanced Acoustic Coupling (MEAC), as provided herein, waves originating from different regions of the transducer will arrive at the intended focal location in-phase. Thus the advantages of MEAC include 1) maximizing the amplitude of the signal at the focus, and 2) minimizing spatial deviation from the intended target location. These improvements serve to both maximize the therapeutic effect and increase the predictability of the treatment location—the latter of which offers major benefits with respect to treatment planning of therapeutic ultrasound.

A MEAC as provided herein includes a specific recipe of coupling liquid medium to match its speed of the sound to the human tissue. Varying concentrations of glycerin alone for glycerin and salt are added to water, and the concentrations depend on the temperature of the water. Using Formulas 1 and 2 described below, the speed of the sound in the MEAC can be matched to the human soft tissue.

A specific example of these improvements has been observed for the in-vivo targeting of porcine muscle with histotripsy. When the histotripsy therapy transducer was coupled to the animal via traditional means—a bolus of degassed water—the resulting bubble cloud was observed to form prefocal to the intended geometric focus of the transducer. However, upon targeting the same location using the MEAC designed to match the sound speed of the porcine tissue path, the bubble cloud formed at the intended geometric focus of the transducer. Furthermore, the transducer driving voltage required for bubble cloud initiation is decreased relative to that required when using traditional water coupling. The reduced driving voltage is because the decreased aberration by using the MEAC.

### Speed of Sound Equations

1. Speed of sound in water c_{W} (m/s) as a function of temperature T (° C.)—Marczak equation [1]:

$${c}_{W} ( T ) = 1.402385 \times 1 {0}^{3} + 5.038813 \times T - 5.799136 \times 1 {0}^{- 2} \times {T}^{2} + 3.287156 \times 1 {0}^{- 4} \times {T}^{3} - 1.398845 \times 1 {0}^{- 6} \times {T}^{4} + 2.787860 \times 1 {0}^{- 9} \times {T}^{5}$$

2. Speed of sound in glycerin c_{G} (m/s) as a function of temperature T (° C.):

$${c}_{G} ( T ) = - 2.2 \times ( T - 25 ) + 1904$$

3. Speed of sound in salt water c_{SW} (m/s) as a function of temperature T (° C.) and salt concentration C (g/L) [2]:

$${c}_{SW} ( C , T ) = {S}_{1} ( C ) + {S}_{2} ( C ) \times ln ( T ) \\ {S}_{1} ( C ) = 1285.9 + 1.7661 \times C - 0.0015 \times {C}^{2} \\ {S}_{2} ( C ) = 62.268 - 0.2197 \times C + 0.0002 \times {C}^{2}$$

Formula 1: Water+Glycerin
1. Volume percentage γ of glycerin in the final mixture:

$${c}_{W} ( T ) \times ( 1 - γ ) + {c}_{G} ( T ) \times γ = {c}_{T} ,$$

where c_{T} is the speed of sound in human tissue:

$$1560 \text{m/s} \to γ = \frac{{c}_{T} - {c}_{W}}{{c}_{G} - {c}_{W}}$$

2. Example Volume Chart

Volume Percentages*

| T (° C.) | Water: (1 − γ) | Glycerin: γ |
|---|---|---|
| 20 | 82.2% | 17.8% |
| 25 (Room Temperature) | 84.5% | 15.5% |
| 30 | 86.9% | 13.1% |
| 37 (Body Temperature) | 89.3% | 10.7% |
| 40 | 91.2% | 8.8% |
| 45 | 92.9% | 7.1% |
| 50 | 94.6% | 5.4% |

*Assume the speed of sound in human tissue is 1560 m/s.

Formula 2: Salt Water+Glycerin
1. Volume percentage γ of glycerin in the final mixture:

$${c}_{SW} ( C , T ) \times ( 1 - γ ) + {c}_{G} ( T ) \times γ = {c}_{T} ,$$

where c_{T} is the speed of sound in human tissue:

$$1560 \text{m/s} \to γ = \frac{{c}_{T} - {c}_{SW}}{{c}_{G} - {c}_{SW}}$$

2. Example Volume Chart

Volume Percentages*

| | Salt Water: (1 − γ) | Glycerin: γ |
|---|---|---|
| C (g/L), T = 25° C. | | |
| 9 (Saline) | 84.3% | 15.7% |
| 18 | 86.2% | 13.8% |
| 27 | 88.3% | 11.7% |
| 36 | 90.4% | 9.6% |
| 45 | 92.5% | 7.5% |
| C (g/L), T = 37° C. | | |
| 9 (Saline) | 88.6% | 11.4% |
| 18 | 90.8% | 9.2% |
| 27 | 93.1% | 6.9% |
| 36 | 95.4% | 4.6% |
| 45 | 97.8% | 2.2% |

*Assume the speed of sound in human tissue is 1560 m/s.
Note:
There is no significant difference in speed of sound between water and saline, so that the volume ratio is similar in both cases.

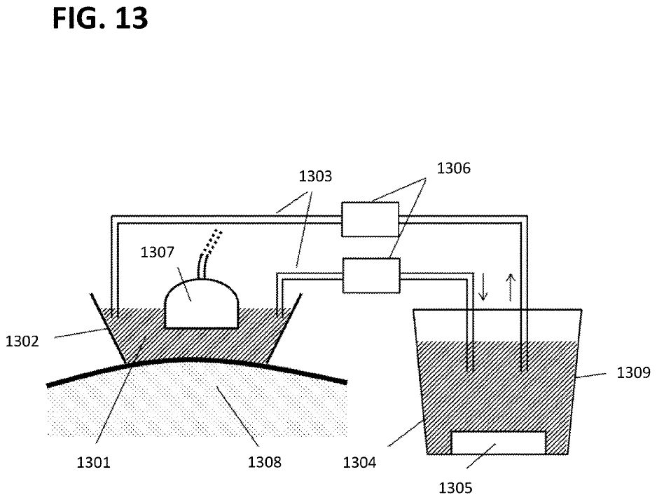

A system is provided in FIG. 13 which includes a fluidics system 1309 configured to deliver a MEAC 1301 according to the concepts described above to a coupling container 1302 of a histotripsy system. Circulation tubes 1303 can pass medium from the fluidics system reservoir 1304 to the coupling container. Degassing pumps 1306 can remove a selected volume or percentage of gas or air from the fluid. The fluidics system 1309 can include a heating coil 1305 or other temperature management system, for managing the temperature of the fluid. When the coupling container is filled with the MEAC, the therapy transducer 1307 can be lowered into the medium to be acoustically coupled to the human subject 1308.

### Mechanical Support Arms and Arm Architectures

In order to support the acoustic and patient coupling system, including providing efficient and ergonomic work-flows for users, various designs and configurations of mechanical support arms (and arm architectures) may be employed. Support arms may be configured with a range of degrees of freedom, including but not limited to allowing, x, y, z, pitch, roll and yaw, as well additional interfacing features that may allow additional height adjustment or translation.

Arms may comprise a varied number and type of joints and segments. Typically, arms may comprise a minimum of 2 segments. In some configurations, arms may comprise 3 to 5 segments.

Arms are also be configured to interface proximally to a main support base or base interface (e.g., robot, table, table/bed rail, cart, floor mount, etc.) and distally to the frame/assembly and overall “UMC” or “coupling solution”. This specific distal interface may further include features for controlling position/orientation of the frame/assembly, at the frame/assembly interface.

For example, in some embodiments, the arm/frame interface may comprise a ball joint wrist. In another example, the interface may include use of a gimbal wrist or an adjustable pitch and roll controlled wrist. These interfaces may be further employed with specific user interfaces and inputs, to assist with interacting with the various wrists, of which may include additional handles or knobs (as an unlimited example), to further enable positioning the UMC/coupling solution. For example, a gimbal wrist may benefit from allowing the frame/assembly to have 3 degrees of freedom (independent of the arm degrees of freedom), including pitch, roll and yaw adjustments.

Support arms, configured with arm wrists, further interfaced with frames/assemblies, may comprise features such as brakes, including cable or electronic actuated brakes, and quick releases, which may interact with one or more axis, individually, or in groupings. They may also include electronic lift systems and base supports. In some embodiments, these lift systems/base supports are co-located with robot arm bases, wherein said robot arm is equipped with the histotripsy therapy transducer configured to fit/work within the enclosed coupling solution. In other embodiments, the support arm is located on a separate cart. In some cases, the separate cart may comprise a fluidics system or user console. In other embodiments, it is interfaced to a bed/table, including but not limited to a rail, side surface, and/or bed/table base. In other examples/embodiments, it's interfaced to a floor-based structure/footing, capable of managing weight and tipping requirements. FLUIDICS SYSTEMS, CONTROL SYSTEMS AND SYSTEM ARCHITECTURES

As a part of overall fluidics management, histotripsy systems including acoustic/patient coupling systems, may be configured to include an automated fluidics system, which primarily is responsible for providing a reservoir for preparation and use of coupling medium, where preparation may include the ability to degas, chill, monitor, adjust, dispense/fill, and retrieve/drain coupling medium to/from the frame/assembly. The fluidics system may include an emergency high flow rate system for rapid draining of the coupling medium from the UMC. In some embodiments, the fluidics system can be configured for a single use of the coupling medium, or alternatively, for re-use of the medium. In some embodiments, the fluidics system can implement positive air pressure or vacuum to carry out leak tests of the UMC and membrane prior to filling with a coupling medium. Vacuum assist can also be used for removal of air from the UMC during the filling process. The fluidics system can further include filters configured to prevent particulate contamination from reaching the UMC.

The fluidics system may be implemented in the form of a mobile fluidics cart.

The cart may comprise an input tank, drain tank, degassing module, fill pump, drain pump, inert gas tank, air compressor, tubing/connectors/lines, electronic and manual controls systems and input devices, power supplies and one or more batteries. The cart in some cases may also comprise a system check vessel/reservoir for evaluating histotripsy system performance and related system diagnostics (configured to accommodate a required water volume and work-space for a therapy transducer).

### Transmit-Receive Electric Driving System

The electric transmit signal to a histotripsy transducer is typically on the order of Kilovolts, while received ultrasound signals typically range from millivolts to tens of Volts. Thus, the transmit-receive electric driving circuitry as described herein is designed and configured to block or heavily attenuate the high-amplitude transmit waveform signals on the order of thousands of Volts, while having sufficient sensitivity and dynamic range to receive the low-amplitude signals on the order of tens of Volts.

Many drive circuitry embodiments and implementations to achieve the stated function/purpose above are described herein. In some examples, the drive circuitry can be retrofitted or added-on to an existing transmit-only histotripsy system to provide transmit-receive capabilities. In other embodiments, the drive circuitry is integrated into an entirely new transmit-receive histotripsy system. Any of the drive circuitry embodiments described herein can be incorporated into or be configured to work with any of the pulse generators and/or amplifiers described herein. The drive circuitry can be disposed or located within the therapy cart of the system, such as cart 110 of FIG. 1A.

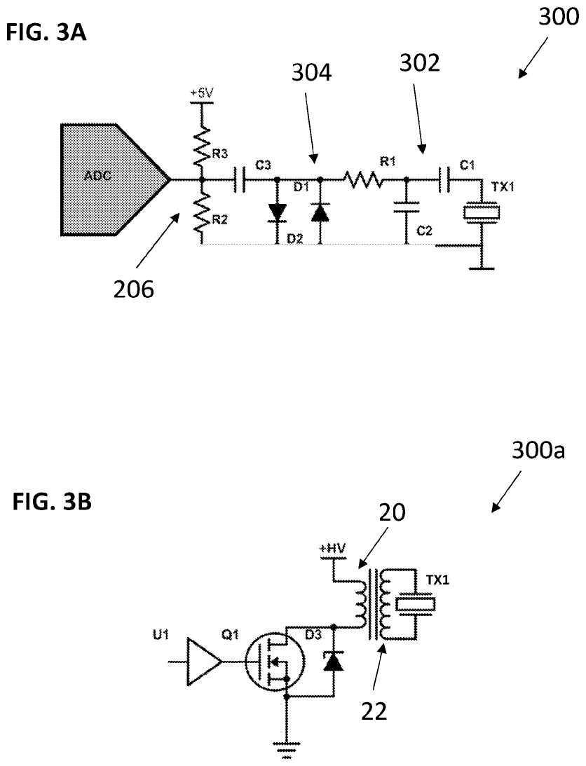

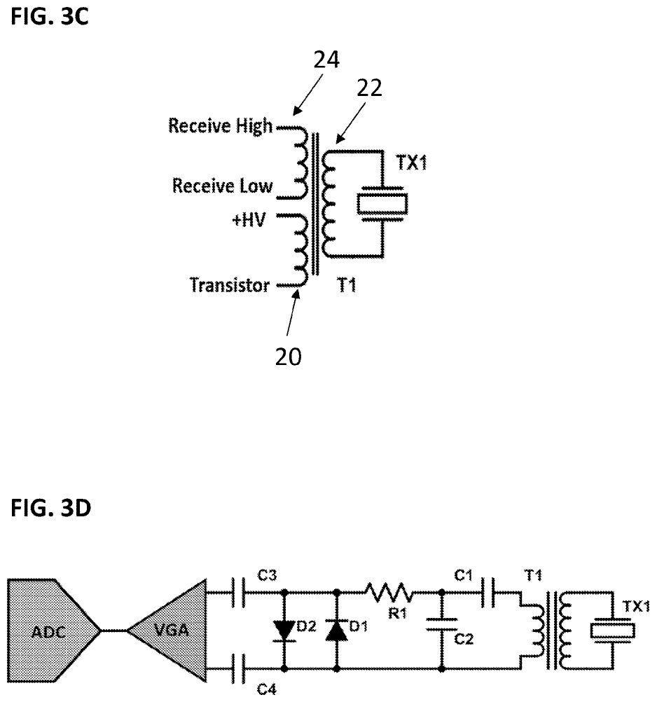

FIG. 3A is one embodiment of a receive drive circuitry 300 configured to be retrofitted onto an existing transmit-only histotripsy system to enable transmit-receive functionality. In the illustrated schematic drawing, a non-linear compressor can attenuate all the signals connected to each of the histotripsy elements, but with more attenuation for the high-amplitude signals and less attenuation for the low-amplitude signals. For example, a capacitive voltage divider 302, as indicated by C1 and C2, can first be configured to attenuate all incoming/received voltage signals from transducer element TX1 to approximately 1-10% (or to attenuate the signals by 90-99%). Then a diode-resistor voltage divider 304, as indicated by D1, D2, and C3, is configured to provide nonlinear attenuation to compress all signals above approximately 1 Volts and alternating current (AC) couple the signal into the analog to digital converter (ADC) for ADC conversion. The final component before the ADC is a voltage level shifter 306, as indicated by R2 and R3, that puts the signal in the appropriate voltage range for the ADC (e.g., typically between +/−0.5V to +/−2V). As described above, this circuitry is configured to be retrofitted to an existing transmit-only histotripsy driving system. For example, separate circuitry boards can be added and connected to the existing transmit circuitry to add the receive functions. In one embodiment, the receive circuitry is added in parallel to the transmit electronics and passively receives signals without affecting the transmit electronics.

FIG. 3B is one embodiment of a drive circuitry 300a that is integrated into high voltage histotripsy driving electronics. In the embodiment of FIG. 3B, a bank of capacitors (not shown) in series with the primary coil 20 of the transformer are charged by a high voltage supply. A driver chip, U1, then triggers the n-channel MOSFET transistor, Q1, which sends a high voltage AC pulse through the transformer primary coil thereby generating an AC pulse in the transformer secondary coil 22 with a voltage proportional to the turn ratio between the coils. The secondary coil can be electrically coupled to each of the transducer elements (in this illustration, transducer element TX1). In one implementation, a turn ratio of approximately 1:3 was used between the primary and secondary coils. This receive drive circuitry is thereby able to generate single-cycle pulses at the center frequency of the transducer on the order of 3 kV. It should be understood that other turn ratios can be implemented.

Referring to FIG. 3C, another embodiment of receive drive electronics for a histotripsy system is shown. As shown, the receive drive electronics can include a secondary transformer coil 22 coupled to the transducer element TX1. Because the driver for this system already includes a transformer at the output of each channel, a third coil 24 can be added to each transformer to be used for the receive electronics, thereby providing total isolation between the driver (e.g., the primary coil 20) and the receiver (e.g., third coil 24). In one implementation, the receive or third coil can be wound with approximately 10-times fewer windings than the secondary transformer coil 22, thereby providing a 10× reduction in voltage between the secondary coil and the third coil. The number of windings on the tertiary or third coil can be tuned for the specific application and need not necessarily be 10-times fewer than the secondary. The ratio depends on the receive signal amplitude and can be adjusted based on desired voltages. In one embodiment, the receive winding (third coil 24) from FIG. 3C can be coupled to a second transformer designed for small signal use with the specifically chosen core material and size such that it would be configured to saturate during the transmit pulses to protect the analog to digital circuitry (ADC) behind it. When receiving signals, however, the second small signal transformer would be configured to not saturate, thereby enabling the appropriate gain and sensitivity for the received signals.

A schematic design of receive circuitry for the integrated receive-capable histotripsy system is shown in FIG. 3D. The primary difference in the embodiment shown in FIG. 3D compared to the embodiment above in FIG. 3A is the transformer, which is described in the embodiment of FIG. 3C. The VGA circuit is added in the embodiment of FIG. 3D, and the “balanced” input with the two capacitors C3 and C4 in series instead of the level shifter as shown in FIG. 3A comprises a digitizer.

In another embodiment, the transmit-receive drive circuitry can include a transmit-receive switch. An integrated drive-receive circuitry with both transmit and receive circuitry on the same board can use a switch to separate the receive signal from the transmit signal. For example, a traditional TR switch with diodes blocks high-voltage transmit signals without attenuating receive signals. A circuit with different linear gain can follow the switch to amplify or attenuate the selected portion of the receive signal properly based on its amplitude to maximize the sensitivity. However, this design would waste a lot of power, be large, and expensive.

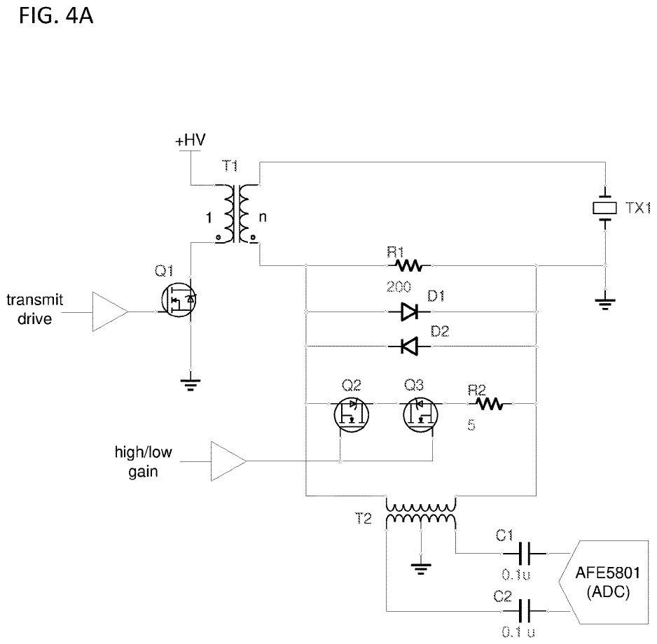

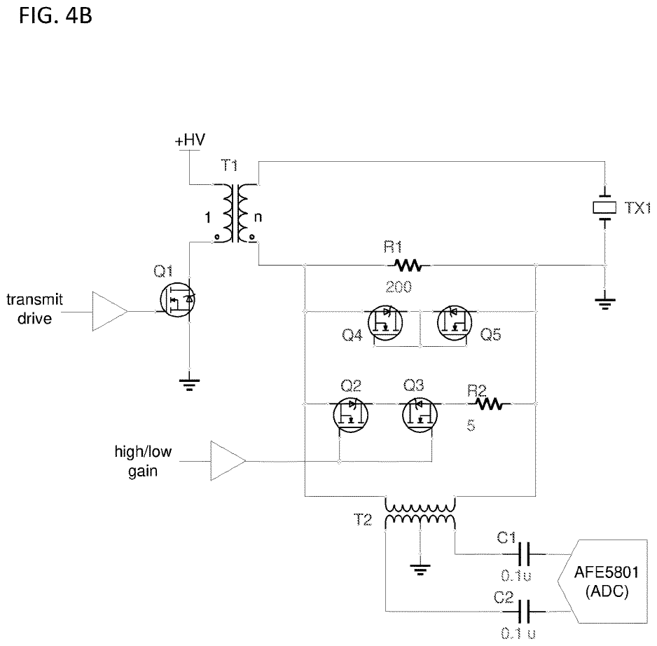

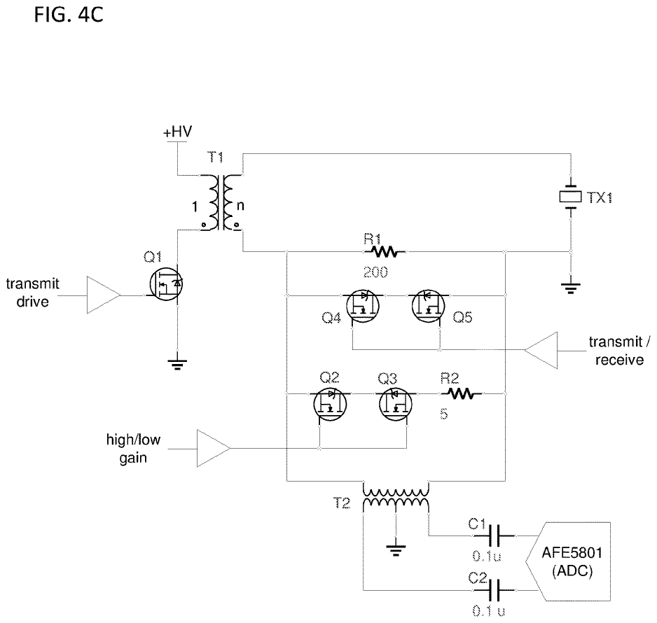

FIG. 4A illustrates another embodiment of drive-receive circuitry that is configured to measure current flowing back from the transducer TX1 through the drive transformer T1 (instead of measuring voltage generated on the transducer during receive as discussed above). The relatively large surface area of therapy transducer array elements compared to a traditional imaging transducer means the transducer array generates a relatively large current, which makes high sensitivity during receive possible, whereas with an imaging transducer, it is only practical to measure the voltage induced by acoustic signals. Normal ultrasound imaging elements would be too small to generate a useable receive current. Therapy elements as described herein are hundreds to thousands of times larger in surface area than traditional imaging elements, so the currents are substantially larger and easy to measure (in the milliamp range rather than microamp). In the circuitry illustrated, current can be measured by a sense resistor in the electrical path (R1). The drive-receive circuitry is configured to pass excess current from large reflections or during the transmit pulse through a set of bypass diodes (D1 and D2). Transmit currents can be as large as 40 A. While the drive-receive circuitry is receiving reflections such as ultrasound reflection signals and/or acoustic cavitation emissions, the sense resistor is configured to measure a current induced in the circuitry by those reflections. Voltage generated across the current sensing resistor is coupled to the ADC through a Balun (T2) and Capacitors C1 and C2. This balanced input configuration is the manufacturer's preferred circuit for the AFE5801 digitizer. Single-ended operation would also be possible for this or other digitizers by directly measuring the voltage on R1 with respect to ground.

The drive-receive circuitry of FIG. 4A can be configured to operate in a low gain mode and a high gain mode. Referring still to FIG. 4A, the circuitry can have two current sensing resistors R1 and R2 so that the overall sensitivity of the circuit can be changed by a large amount. As shown, this can be implemented with a pair of transistors Q2 and Q3 that are configured to switch on/off a small value resistor R2 (low sensitivity) in parallel with the larger value resistor R1 (high sensitivity). The resistance of the circuit can be changed very rapidly with these transistors to enable the use of both the low setting over part of a received burst of data (e.g., a received signal with a higher amplitude such as ultrasound reflection signals from bones) and the high setting a few microseconds later (e.g., a received signal with a lower amplitude such as acoustic cavitation emission signal from cavitation collapse). Because the sensor is directly changed, both scales have very high SNR unlike a variable gain amplifier where the SNR is usually worse for higher gain. In some embodiments, additional sense resistors can be implemented in the same manner for even wider dynamic range. For the circuit shown in FIG. 4A, the high gain mode is configured to measure currents up to 5 mA in the ADC which is coupled to the circuitry via transformer T2, while the low gain mode is configured to measure currents up to 200 mA in the ADC.

FIG. 4B shows an alternate embodiment where instead of bypass diodes, low gate threshold MOSFET transistors Q4 and Q5 can be implemented for passing the large transmit currents. With the advances in transistors, there are now transistors that are smaller, cheaper, and higher performance than any diode for this bypass role. These transistors can have a higher turn on voltage than a single diode, which allows the use of the full dynamic range of the ADC more easily.

FIG. 4C shows a third embodiment where the bypass transistors Q4 and Q5 are explicitly controlled as an active transmit-receive switch. The transistor gates are connected to a gate drive signal to force the transistors fully on (for transmit mode) or fully off (for receive mode) which could be +/−5 V, for example, depending on the transistor drive requirements. This configuration may reduce RF noise generated during transmit where instead passively switched bypass components must turn on and off rapidly at the frequency of the ultrasound. This design has a tradeoff of a minor increase in complexity.

The analog received signals described above can be converted to digital signals and then collected and processed. The signal received from the histotripsy transducer array can be, for example, reflections from bones or soft tissue or acoustic emission signals from cavitation. These signals are typically received in a specific time window after the histotripsy pulse (e.g., tens to hundreds of microseconds after transmission of the therapy pulse(s)). Thus, the hardware and software described herein is configured to synchronize the time clock of transmit, receive, and ADC conversion and sampling to obtain the appropriate time window after each histotripsy pulse that contains the desired received signals. If the synchronization and time window is set properly, then the desired received signals can be collected and processed.

To achieve proper synchronization and time windowing, any of the transmit-receive drive electronics described herein can include an embodiment in which a single field-programmable gated array (FPGA) device connected to the ADC can be used to control both the transmit and receive operations of the transducer, as well as the ADC for some subset of or all channels of a histotripsy system. By providing the FPGA with a single clock off of which the timings of the operations to be executed by the separate subsystems are based, synchronization between subsystems can be guaranteed especially when multiple FPGAs are used to control various subsets of histotripsy transducer elements. Setting the appropriate time window to receive the signals can then be achieved through appropriate assignment of the timings of the respective operations when programming of the FPGA. In cases where multiple FPGAs are required, for instance in arrays with too many transducer elements to control from a single device, a single clock line can be fanned out to all of them for synchronization, and a centralized ‘master’ FPGA can be used to trigger the execution of their operations within the appropriate time window.

Alternatively, any of the transmit-receive driving electronics described herein can include multi-FPGA systems can be setup to run in a ‘headless’ mode wherein no centralized ‘master’ FPGA is required to issue/fan out a single shared clock line or trigger the execution of individual boards' operations. In such a mode, each FPGA would be set to run off of its own individual clock and to monitor and update two common ‘program-execution-state’, and one common ‘execute-operation’, open-drain hardware IO lines shared by the whole system. The open-drain lines operate such that, if any single FPGA applies a low signal to the lines, the signal measured anywhere on the line would register low; if and only if all FPGAs apply a high signal to the lines, the signal measured everywhere on the line would register high. The two ‘program-execution-state’ lines would be used to the FPGAs to issue system-wide 1) ‘ready-to-execute’ and 2) ‘done-executing’ signals and by default each FPGA would apply a low signal to each of these lines; each FPGA would apply a high signal to the ‘execute-operation’ line. While running a program, upon reaching a new executable instruction in the program, each FPGA would update the ‘ready-to-execute’ line to apply a high signal to it, and enter a wait state wherein it would monitor the signals on both the ‘ready-to-execute’ line and the ‘execute-operation’ lines. Once all FPGAs reached the ‘ready-to-execute’ state, the signal registered on the ‘ready-to-execute’ line would become high; the first FPGA in the system to detect a high state on the ‘ready-to-execute’ line would issue a low signal on the ‘execute-program’ line causing it to register low everywhere. Upon detection of the low signal on the ‘execute-program’ line, each FPGA would set the value on its own terminal of the ‘execute-program’ line to be low and execute its stored commands. Once each FPGA finished running its respective commands, it would apply a high signal to both the ‘done-executing’ and ‘execute-program’ lines. Once both the ‘done-executing’ and ‘execute-program’ lines registered high, the FPGAs would reset all of the shared open-drain line values to their defaults, load the next instruction in the program, and repeat the process for each instruction until the program was completed.

A fully connected set of receiving elements can generate large amounts of data, so strategies to reduce the data load are proposed to allow acquired signals to be transferred and processed in real-time to meet the monitoring needs during therapy. These strategies can be applied to any of the transmit-receive driving electronics described herein. Such strategies may include, for example, artificially down sampling the incoming data from the ADC in the firmware running on FPGA (e.g., by storing only every other data point generated by the ADC, or the average of the data points generated across multiple acquisition cycles). This effectively reduces the sampling frequency, thus reducing the data load, but doesn't sacrifice temporal precision or dynamic range or result in an increase in noise in the system. Differential compression schemes, wherein all data captured after the first time point is stored as the difference between the values captured at adjacent time points may also be applied. For example, for a value at time 1 of X, and at time 2 of Y, one could store the value of the difference between Y and X, D=Y−X, at time 2 instead of the value of Y directly, and then calculate the actual value of Y during processing as Y=X+D. In this way, nominal values of say X=64000 and Y=63900, which combined represent 4 bytes of data, could be stored as X-64000 and D=−100, which combined represent 3 bytes of data and allow the full recovery of the value of Y. As the length of the data record gets longer, this compression strategy results in data reductions proportional to ratio of the size of the variable needed to store the difference value compared to the size of the variable required to store the actual value, which can generally reduce data loads in the current system by 30%-50%, but could result in significantly larger reductions in systems where the individual data elements are larger in size. In applications not demanding real-time processing/compression, further reductions in data size can be achieved through frequency domain transforms using methods similar to those employed to compress audio files.

As some of the events that need to be monitored during histotripsy therapies will require very high temporal precision (e.g., the signals from individual cavitation events), while others will require little precision (e.g., the reflections of signals off of large boundaries, e.g., the skull, ribs, tissue interfaces), strategies to dynamically alter the compression ratio can be implemented to fully utilize the incoming data for real-time applications. To that end, the firmware and software that control the data acquisition have been configured such that the sampling frequency and compression strategy used during acquisition can be set on a per-channel basis in the array, and can be independently updated in real-time, even in the middle of an individual acquisition event. This allows for different sub-apertures of the array to be set up to monitor different features of the therapy at the requisite sampling frequency and compression settings, as well as for the receive system to be set to the maximum sampling frequency/minimum compression settings across all elements of the array as needed to monitor short-lived events with potentially weak signals, and then set back to lower sampling frequencies with higher compression settings outside the window requiring maximal monitoring. This allows short-lived events of this type to be fully monitored without necessitating cut-offs in the acquisition to reduce the data load which could otherwise potentially result in reducing the physical size of the actively-monitored field or drastic reductions in monitoring speed during therapy.

In some situations, the receive signal amplitude may be low and the noise may be high, resulting in a low signal-to-noise ratio (SNR). One method to reduce the noise and increase SNR is to oversample and average in firmware (e.g., FPGA firmware) before storing data. This also helps increase dynamic range and reduces memory requirements. Another technique is to implement a dynamic variable sample rate. For example, the ADC can be configured to always run at 50 MHz, but high time precision may only be needed over certain portions of the data record. In the portions of the signals where such a high frame rate is not needed, samples can be decimated or averaged to greatly reduce storage requirements.

The bandwidth of the therapy transducer elements is typically low, but a high sampling rate can be used for sampling for good timing precision. Receive data should compress exceptionally well in the Fourier domain (at least a factor of 10, maybe a lot more). The FPGAs can be configured to perform this compression before storage or transmit either in firmware or in software. Data compression is the key to implementing real time monitoring, the system will be overwhelmed by the amount of receive data collected.

In applications where real time monitoring is not essential, or where treatment speed needs to remain higher than possible while simultaneously transferring the full acquired signals to the user's computer after each pulse, the system can be configured to transfer only partial signals and/or store the acquired signals directly on the FPGA devices themselves for transfer to the control computer later. This would allow uninterrupted acquisition of signals from all delivered pulses without limiting treatment speed. Such capabilities are useful for monitoring long-term changes in acquired signals. For example, there is inherent variability in acoustic cavitation emission (ACE) signal features associated with the ablative state of the targeted tissues that make the tissue state difficult to track pulse-to-pulse, but characteristic changes in the ACE signals exist over longer treatment time scales (e.g., >20 applied pulses) that allow the ablative state of the tissue to be assessed. One could transfer partial signals in real time to allow localization and mapping of the cavitation events on a per-pulse basis, while storing larger-record length signals on the FPGA to be transferred intermittently to assess the state of the ablation in the therapy target.

In some situations, it is possible to generate focal pressures far in excess of twice what is nominally required to generate cavitation during therapy and in such cases, it may be possible to generate cavitation using fewer than half of the histotripsy transducer array elements. The software controlling the histotripsy array allows for the elements of the array to be easily partitioned into independently controllable sub-apertures, effectively allowing a single physical histotripsy transducer array to be operated as multiple separate histotripsy arrays. In this way, multiple locations within the focal volume can be targeted for treatment concurrently using the separate sub-apertures of the array, allowing for increases in treatment speed without necessitating an increase in the rate at which pulses are delivered.

### Ultrasound-Based Real-Time Histotripsy Dose Monitoring

Different tissue types/components require different histotripsy doses to completely disrupt. For example, tissue with a higher mechanical strength (e.g., vessels, nerves) takes a higher dose of histotripsy to destroy. Tissue that is mainly cellular vs. collagen can take different doses to destroy. It takes a much higher dose to destroy the extracellular matrix of tissue compared to cellular components. Tissue can be heterogeneous, thus even within a target tissue, it can take different dose to treat a one location of the target issue versus the other location (e.g., gray matter vs. white matter in the brain). In addition, histotripsy dose has an impact on the immune response, as different tumors require different histotripsy doses for complete ablation or maximizing the immune response. Even within a tumor, it is possible to have different tissue (cells vs. extracellular) components. According to one aspect of the disclosure, systems and methods for real-time monitoring of histotripsy dosage delivery and damage or treatment of tissue both spatially and temporally is provided to enable tracking of treatment progress and the desired damage (e.g., homogenous ablation of the target tissue or ablation of one tissue type while preserving another within the target tissue). In one aspect, the spatial and temporal monitoring of histotripsy treatment progress is performed with real-time monitoring of ultrasound signals generated during therapy. In other aspects, other imaging modalities such as MRI can be used to monitor histotripsy treatment progress. Alternatively, ultrasound monitoring co-registered with MRI scans showing the target tumor can be used to monitor histotripsy treatment progress.

According to aspects of the disclosure, real-time histotripsy dose monitoring can be used to 1) identify and locate cavitation formation within a target tissue volume, 2) identify when cavitation results in complete cellular disruption, 3) identify various tissue types and their locations within the target tissue volume, and 4). In some aspects, the real-time histotripsy dose monitoring is performed not with real-time imaging, but instead by monitoring and analyzing acoustic cavitation emission (ACE) signals received with a transmit-receive histotripsy array. It is noted that these signals are non-imaging data, but instead convey features relating to the formation and collapse of cavitation within the tissue volume, which can then be used to determine the tissue type and/or the extent of damage to the tissue caused by cavitation (e.g., the extend of cellular disruption).

Real-time ultrasound feedback received from the transmit-receive capable histotripsy array can monitor the dose delivery and damage spatially and temporally. After transmitting histotripsy pulses into a focal location within a target tissue region with a transmit-receive histotripsy array to generate cavitation, the cavitation nucleation, expansion, and collapse signals can be detected via the acoustic cavitation emission (ACE) signals received by the transmit-receive histotripsy array. The received ACE signals can then be processed by the system to quantitatively monitor the nucleation, expansion, and collapse of cavitation, as well as tissue treatment progression and completion. Many different parameters or features of the ACE signals can be used for this purpose, including but not limited to timings and amplitudes of the cavitation bubble expansion signals, collapse signals, rebound signals, cavitation collapse time (i.e., the time between the expansion signal and collapse signal), peak amplitude of the expansion signal, peak amplitude of the collapse signal, amplitude ratios of the growth and collapse ACE signals, or the decay rates of the rebound-associated ACE signal amplitudes.

In some aspects, the ACE signals can be evaluated or processed to determine or identify the tissue type at the cavitation focus. Different tissue types respond differently to cavitation, and the ACE signals resulting from this cavitation reflect those tissue type differences. For example, soft, cellular, or fatty tissues such as white matter tissue, cellular tumors, or fat may result in cavitation with high cavitation lifespans, compared to stiff or fibrous tissues like neurovascular bundles or fibrous tumors which may have much shorter cavitation lifespans comparatively. Correspondingly, the histotripsy dose required to completely disrupt the fibrous tissues is higher compared to that required to completely disrupt the cellular tumor or normal liver. The histotripsy dose required to completely disrupt gray matter in the brain is slightly higher compared to that required to completely disrupt the white matter.

In some aspects, these ACE signals can be used to determine if cavitation is being formed in a targeted tissue type within the target tissue volume (e.g., within a fibrous tumor tissue growing in white matter of the brain) or if cavitation is being formed in a non-targeted tissue type within the target tissue volume (e.g., within gray matter of the brain). For example, the brain typically consists of gray matter which contains high concentration of neuronal cell bodies and white matter which contains myelin and is an insulating layer or sheath around nerves. Brain tumor growth can occur in white matter, in gray matter, or where the white matter and gray matter meet. Regardless of the location of the tumor, treatment of a brain tumor can include monitoring ACE signals to determine the tissue type at the focal location and guide the therapy to provide complete cellular disruption in only the desired tissue types (e.g., tumor tissue and/or white matter tissue) and to avoid damaging un-desired tissue types (e.g., gray matter).

Knowledge of the tissue type at the focal location derived from the ACE signals can be used to spatially map the tissue types within a target tissue volume. Additionally, this mapping or spatial awareness of where different tissue types are located can be used to adjust or modify a treatment plan, including adjusting or modifying histotripsy pulse waveforms, amplitudes, and pulse repetition frequency (PRF) based on the tissue type. For example, when treating brain tissue, test or sample pulses may be directed into various locations within the tissue volume, and resulting ACE signals may be evaluated to determine the tissue type (e.g., such as by monitoring cavitation lifespan of the resulting cavitation). The system, with knowledge of various cavitation lifespans for an assortment of tissue types, may be configured to identify a tissue type for each focal location. Since different tissue types also have different cavitation thresholds (e.g., the pressure required to generate cavitation), then the amplitudes or pulse sequences for each treatment location within a tissue volume can be adjusted so as to produce cavitation within desired tissue types and to not produce cavitation (or to produce cavitation lifespan below a threshold) within other tissue types. For example, if treating a tumor that spans or is near both white matter and gray matter tissue, the amplitude of the histotripsy pulses may be adjusted or modified such that they generate cavitation in the tumor and white matter tissue while not generating cavitation in the gray matter tissue (e.g., assuming that the gray matter tissue requires a higher cavitation threshold than the white matter tissue and tumor) or while generating a cavitation lifespan below a threshold to minimize the damage in the gray matter (our preliminary data shows that the cavitation lifespan is shorter in the gray matter than in the white matter even using the same histotripsy parameters).

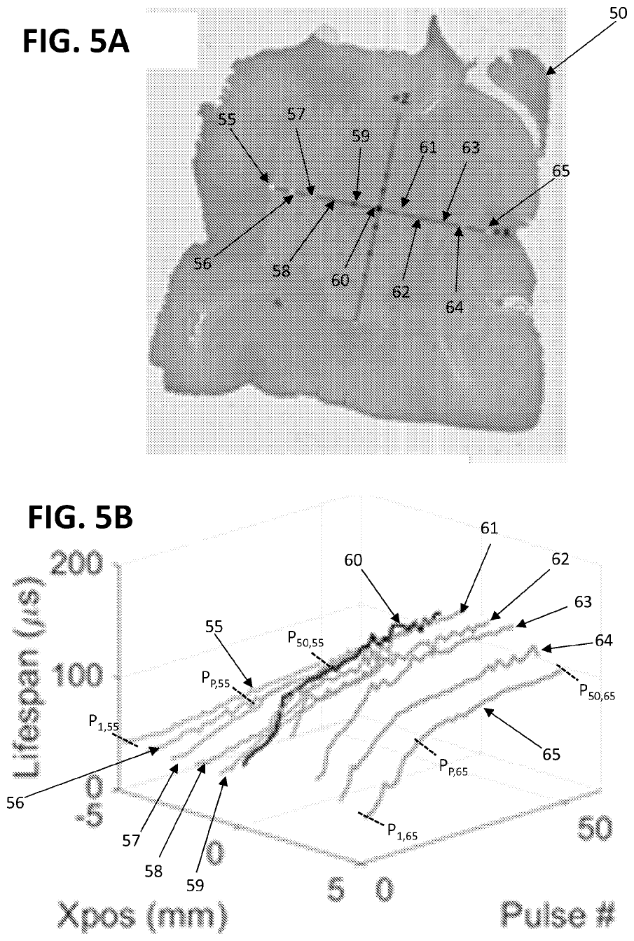

FIGS. 5A-5B show one example of monitoring ACE features with a transmit-receive transducer array to quantify histotripsy-induced tissue damage spatially and temporally. In FIG. 5A, a target tissue volume 50 can comprise brain tissue having both white matter and gray matter. White matter is typically found closer to the center of the brain, and gray matter is typically found in the outer cortex. It can be difficult to distinguish between white and gray matter under real-time imaging, particularly since ultrasound imaging is difficult or impossible through the skull. In FIG. 5A, the target tissue volume 50 has been individually treated at treatment locations 55-65 along the x-axis (relative to the transducer) as shown. In this example, treatment locations are positioned in 1 mm increments from −5 mm to 5 mm along the x-axis. Each treatment location 55-65 represents a location within the target tissue volume 50 that receives a dose of histotripsy energy resulting in cavitation to mechanically lyse the tissue at that treatment location. In some embodiments, the transmit-receive transducer array can be mechanically moved, e.g., with the robotic arm or robotic positioning system, between each of the treatment locations. In other embodiments, the transmit-receive transducer array can be electronically steered (e.g., phased array steering) between the locations. In additional embodiments, movement between the treatment locations can be a combination of mechanical movement and electronic focal steering. As the histotripsy pulses are delivered to each of the treatment locations in FIG. 5A, the transmit-receive transducer array can be configured to detect ACE signals from the cavitation to monitor the treatment both spatially and temporally.

In some aspects, the precise location of the transmit-receive transducer array is known from the position and orientation of the robotic arm and the robotic navigation system. The position of the focus for each focal location is also known from the natural focus distance (or electronically steered focal location) relative to the transducer. Therefore, each focal location can be spatially mapped in the system and associated with the received ACE signals for each focal location. This information can be presented to a user, and can also be overlaid upon other images of the target tissue volume including ultrasound, CT, or MRI imaging. When ACE signals are used to determine a tissue type at a specific focal location, this information can also be presented to the user.

FIG. 5B shows plots of the cavitation lifespan at each of the treatment locations 55-65 (x-pos—spatially) vs. histotripsy pulses (doses-temporally) applied, with 50 pulses applied at each location. As shown in each of the individual plots, the cavitation lifespan as derived from the ACE signals (time between cavitation nucleation and collapse) increases starting at pulse P_{1,65} and plateaus with increasing number of histotripsy pulses applied (or time) at plateau pulse P_{P,65}. As shown, the cavitation lifespan remains relatively constant or steady between plateau pulse P_{P,65} and pulse P_{50,65}. Contrast the cavitation lifespan plot associated with treatment location 65 with that of treatment location 55. As shown in FIG. 5B, the cavitation lifespan for treatment location 55 increases between P_{1,55} and plateaus at plateau pulse P_{P,55}. As shown, the cavitation lifespan remains relatively constant or steady between plateau pulse P_{P,55} and pulse P_{50,55}. Complete cellular disruption is associated with the plateau of the cavitation lifespan, therefor, for treatment locations 55 and 65 complete cellular disruption is accomplished at plateau pulses P_{P,55} and P_{P,65}, respectively. The biggest difference between the plot associated with treatment location 55 and that of treatment location 65 is that the cavitation lifespan plateaus later at treatment location 55 than it does at treatment location 65.

To provide additional context, when the treatment locations of FIGS. 5A-5B are spatially mapped to the brain tissue, treatment locations 55 through 59 fall within gray brain matter tissue, and treatment locations 60 through 65 fall within white brain matter. Collectively, referring to FIG. 5B, it can be seen that all the treatment locations within the gray brain matter (e.g., treatment locations 55-59) require more histotripsy pulses for the cavitation lifespan to plateau compared to the treatment locations in white matter (e.g., treatment locations 60-65), which require fewer histotripsy pulses before the cavitation lifespan plateaus. Therefore, ACE-derived cavitation lifespan can be used by the system to monitor histotripsy treatment both spatially and temporally and further to quantify and localize damage specific for a tissue type (e.g., gray matter vs. white matter). While gray and white matter tissues are similar, gray matter is slightly stiffer than white matter tissue. In the context of the ACE map of FIG. 5B, it makes sense that gray matter tissue would have slightly lower maximal cavitation lifespans and require more treatment before plateauing (indicating complete cellular disruption) compared to white matter tissue.

In some embodiments, the system can be configured to deliver histotripsy pulses into a target tissue volume to form cavitation at a treatment location within a target tissue and receive ACE signals from the cavitation. Features derived from the ACE signals, such as maximal cavitation lifespan or time/pulses until cavitation lifespan plateau, can then be used by the system to determine the tissue type and/or to determine treatment progress or completion. In some embodiments, histotripsy pulse delivery can be immediately and automatically stopped when the system identifies the plateau, and therefore treatment completion or cellular disruption, to avoid delivering excessive ultrasound pulses into the tissue location. In some embodiments, treatment completion or complete cellular disruption can be indicated to the user, in the form of a visual indicator (e.g., a warning or message on a console or GUI of the system) or as an audible sound or alert. In some embodiments, the tissue type determination is based upon the amount of pulses delivered into a given treatment location (or time) before the cavitation lifespan as derived from received ACE signals plateaus or stops increasing. Alternatively, the plateau can be defined as a change in the rate of increase of the cavitation lifespan. For example, histotripsy therapy may result in cavitation having a cavitation lifespan that increases at a first cavitation lifespan rate until a plateau, where subsequent pulses either stop increasing, or increase at a second cavitation lifespan rate that is lower (or substantially lower than) the first cavitation lifespan rate.

Referring back to FIG. 5B and focal location 65, pulses delivered between P_{1,65} and P_{P,65} result in a cavitation lifespan that increases at a first cavitation lifespan rate, and pulses delivered between P_{P,65} and P_{50,65}, after the plateau, increase at a second cavitation lifespan rate or do not increase at all. Additionally, the tissue type determination can be based simply on the maximal value of the cavitation lifespan. High maximal cavitation lifespans within a first cavitation lifespan range may indicate fatty or healthy cellular tissues such as white matter, while lower maximal cavitation lifespans within the first cavitation lifespan range may indicate fibrous materials such as pancreatic tumors. Put another way, maximal cavitation lifespans above a threshold may indicate cellular tissues while maximal cavitation lifespans below the threshold may indicate stiff or fibrous tumors. It is noted that not all healthy tissue is softer than tumors, since many tumors are not stiff or fibrous. To provide a specific example, maximal cavitation lifespan in white and gray matter is fairly similar, typically ranging between 30-50 μs (with gray matter being slightly stiffer and therefore having a lower maximal cavitation lifespan) in the first cavitation event and between 100-120 μs when plateaued, with gray matter having a slightly lower maximal cavitation lifespan. Fibrous tumors, on the other hand, may have maximal cavitation lifespans 2-3× or less. To produce a larger difference in the cavitation lifespan between different tissue types, a higher peak negative pressure or a slightly longer pulse duration can be used.

In some aspects, the system can be configured to automatically determine a tissue type at a focal or treatment location based on ACE features and or thresholds associated with received ACE features. In additional aspects, the system can be configured to determine if the focal or treatment location is positioned within a fibrous tissue or if the focal or treatment location is positioned in a cellular or fatty tissue. In some embodiments, the system can access or include a database or lookup table that correlates various ACE features with specific tissue types to make the tissue type determination. For example, the system may classify fibrous tumor tissues (or other fibrous tissues) as being associated with ACE signals that result in generally lower maximal cavitation lifespans or higher dose/time required for cavitation lifespan plateau than that those of surrounding cellular tissues which generally have higher maximal cavitation lifespans or lower dose/time required for cavitation lifespan plateau. To provide a specific example, if it has been determined that, for a given histotripsy pulse sequence, complete cellular disruption of tumors occurs when the cavitation lifespan plateaus within a first threshold range, complete cellular disruption of gray brain matter occurs when the cavitation lifespan plateaus within a second threshold range, and complete cellular disruption of white matter tissue occurs when the cavitation lifespan plateaus within a third threshold range, then the system can monitor the cavitation lifespan from received ACE signals, determine when the cavitation lifespan plateaus, and make a tissue-type determination.

Additionally, as described above, the maximal cavitation lifespan itself can provide an indication as to the tissue type. Generally, fibrous tissues such as fibrous tumors will have a lower maximal cavitation lifespan than healthy surrounding tissues. As such, the system can be configured to automatically detect when the focal location or cavitation is positioned within a tissue type that is not to be treated vs. when the focal location or cavitation is positioned within a tissue type that is to be treated (e.g., tumor tissue) based on the maximal cavitation lifespan. Additionally, if the system is moving the focal zone of the transducer through a tissue volume, and the received ACE signals indicate a large decrease in cavitation lifespan, the system can make a determination that the focal zone has moved from a tissue to be treated (e.g., a tumor, with a relatively higher maximal cavitation lifespan) into a tissue that is not to be treated (e.g., healthy surrounding tissues, with a much lower maximal cavitation lifespan). In some embodiments, the system can be automatically configured to stop or pause treatment or histotripsy pulse delivery (e.g., with the pulse generator), or alternatively, to move the focus of the transducer back towards the previous location (e.g., back to within the tumor tissue with the robotic positioning system).

### Selective Treatment of Brain Tissue

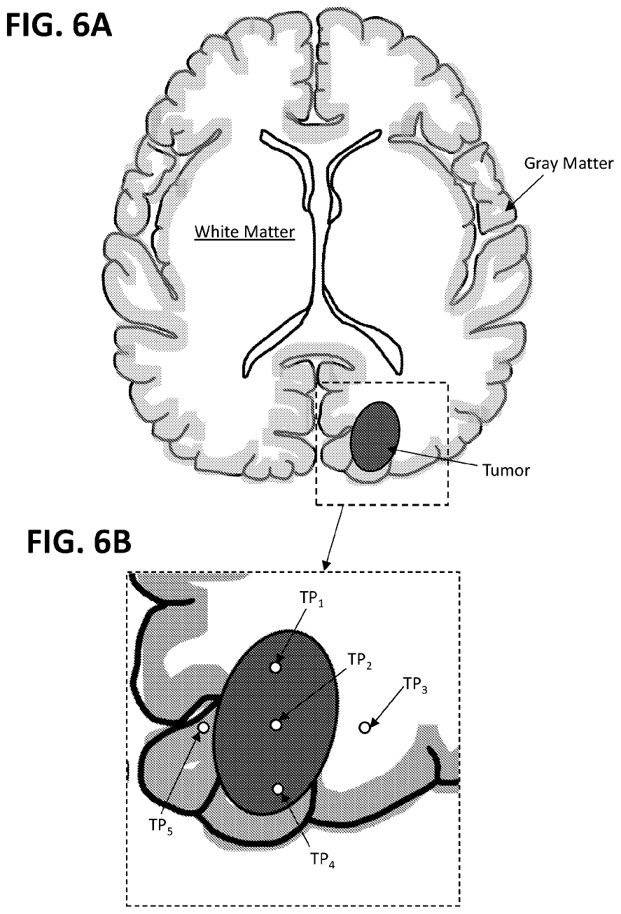

FIG. 6A shows a cross-sectional view of a subject's brain, including white matter, grey matter, and a fibrous tumor to be treated with histotripsy therapy. FIG. 6B is a close-up view of the tumor including surrounding white and grey matter regions. In FIG. 6B, histotripsy pulses may be delivered to various test points TP1 through TP5 in the tissue. ACE signals resulting in cavitation formed at the test points can be used to determine the tissue type at each test point. For example, maximal cavitation lifespans, or the number of pulses or time to reach cavitation lifespan plateau at TP3 may be evaluated by the system to determine that the tissue at TP3 is or is likely to be white matter tissue. Similarly, maximal cavitation lifespans, or the number of pulses or time to reach cavitation lifespan plateau at TP5 may be evaluated by the system to determine that the tissue at TP5 is or is likely to be grey matter tissue. Additionally, maximal cavitation lifespans, or the number of pulses or time to reach cavitation lifespan plateau at TP1, TP2, and TP4 may be evaluated by the system to determine that the tissue at TP1, TP2, and TP4 is or is likely to be fibrous tumor tissue.

As described above, the tissue types at various focal locations (or test points) can be spatially mapped and presented to a user of the system. Knowledge of the location of various tissue types can also be used to adjust or modify a treatment plan of a target tissue volume. For example, the robotic positioning system can be controlled to avoid delivering therapy to tissue locations that include non-targeted tissues (e.g., grey matter and white matter). Additionally, knowledge of the tissue type can be used to adjust or modify pulse parameters or amplitudes to ensure that the cavitation threshold necessary for complete cellular disruption is achieved. Additionally, sensitive tissues such as gray matter can be protected by adjusting the amplitude or pulse sequence to generate cavitation in tissues such as tumors or white matter without generating cavitation in grey matter. Therefore, knowledge of tissue type as derived from ACE signals can be used to selectively treat targeted tissues without damaging non-targeted or un-desired tissues.

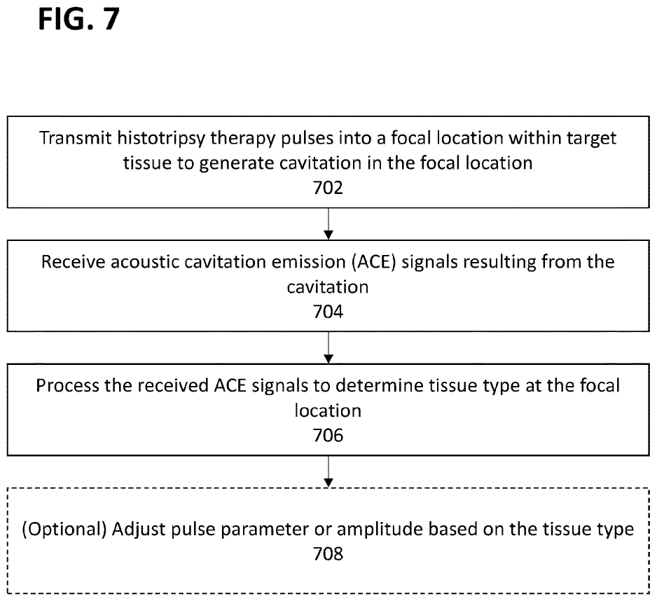

For ACE-based tissue detection, a specialized method and algorithm illustrated by the flowchart in FIG. 7 can be implemented in a histotripsy system of the present disclosure. The method can be a computer implemented method, or an algorithm or algorithms executed by a processor of a histotripsy system to cause the histotripsy system to perform the following operations: At step 702, the method can include transmitting histotripsy therapy pulses into a focal location within target tissue with an ultrasound transducer array to generate cavitation in the focal location. In some aspects, transmitting the histotripsy therapy pulses can be for the purposes of testing the cavitation response at a particular focal location. In other embodiments, the pulses are transmitted with the intent to treat the focal location to cause complete cellular disruption. As described above, a plurality of transducer elements of the array can each transmit histotripsy pulses into the tissue. The ultrasound transducer can be mounted on a robotic arm and configured to deliver histotripsy pulses to one or more treatment or focal locations within a target tissue to generate cavitation at each focal location.

Next, at operation 704, the method can include receiving acoustic cavitation emissions (ACE signals) resulting from the histotripsy-induced cavitation. The receiving of ACE signals can utilize, for example, any of the systems or drive electronics described above. The histotripsy system can process or analyze the ACE signals to extract or identify features of the ACE signals, including but not limited to timings and amplitudes of the cavitation bubble expansion signals, collapse signals, rebound signals, cavitation collapse time (i.e., the time between the expansion signal and collapse signal), peak amplitude of the expansion signal, peak amplitude of the collapse signal, amplitude ratios of the growth and collapse ACE signals, or the decay rates of the rebound-associated ACE signal amplitudes.

Next, at an operation 706, the method can use the information or features encoded in these ACE signals (e.g., maximal cavitation lifespan, time/pulses to reach cavitation lifespan plateau, etc.) to determine a tissue type at the focal location. In some aspects, the system can access or know features which features or ranges/values of features are associated with specific tissue types. For example, the system can access a database or lookup table of ACE features for fatty tissues, healthy tissues, fibrous tissues, tumor tissues, etc., and compare the received ACE features to the database or lookup table to determine the tissue type. For example, relatively high maximal cavitation lifespans (e.g., on or above 90-100 μs) may indicate a fibrous tissue such as a tumor, while lower cavitation lifespans (e.g., on the order of 30-50 μs) may indicate healthy or non-targeted tissues such as gray or white matter.

Finally, at operation 708, the method can optionally include adjusting one or more parameters of the driving electric signal to each array element account for the tissue type detected at that focal location. For example a treatment plan may be modified to avoid moving the focus of the transducer to tissues that are not to be treated (such as white/grey matter), or alternatively, to adjust an amplitude of pulses such that cavitation is not generated in these tissues (e.g., the pressure is below the cavitation threshold).

### Selective Treatment of Fat (Fat Removal)

The principles described above can also provide systems and methods of using histotripsy for fast reduction of fat without damaging the surrounding tissue (including vessels, nerves, muscles, etc.). Obesity is a major health issue, increasing the risk of many diseases including cardiovascular diseases, cancer, etc. There are millions of Americans suffering from health complications associated with a high fat level. The current standard surgical treatment for fat reduction of subcutaneous fat is invasive. Other alternative methods include laser- or ultrasound-based approaches with limitations on slow treatment speed and small treatment volume.

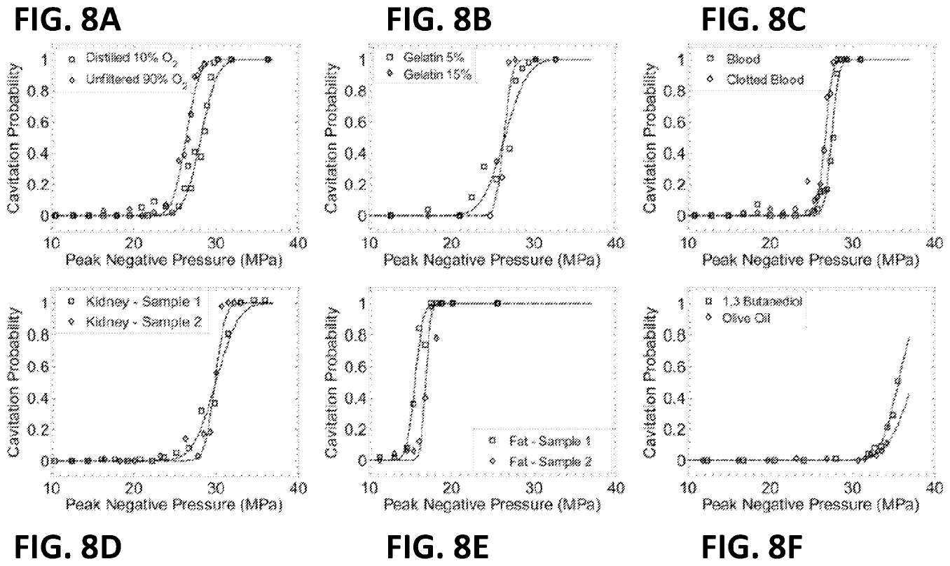

Histotripsy uses microsecond ultrasound pulses to liquefy the target tissue into acellular debris by controlling acoustic cavitation, which is an entirely different mechanism from the current approaches. The generation of cavitation during histotripsy is achieved when microsecond length pulses reach negative pressures that exceed an intrinsic threshold and overcome the surface tension of pre-existing nanometer gas pockets in the tissue. This threshold has been measured to be p- of 26-30 MPa for water-based tissues (such as nerve, vessel, liver, kidney, heart, brain) and 14 MPa for fat, when using 1-cycle pulses. FIGS. 8A-8F show probability data and fit curves for each sample type tested. Each data point is the fraction of 100 pulses where cavitation was detected. Curves were fit by nonlinear least squares regression to each data set.

By using 1-cycle pulses and a peak negative pressure above 14 MPa and below 26 MPa, histotripsy can be used to non-invasively and selectively liquefy the fat that can be absorbed by the body via metabolism or be removed with a small catheter, while keeping the surrounding other tissue intact, including nerves, vessels, and muscle.

Histotripsy has the potential to reduce large volume of subcutaneous or internal fat (e.g., in the abdomen, legs, arms) as well as precise fat reduction for face or body sculpturing. To accelerate the fat reduction treatment, a histotripsy transducer with a large focal volume (>5 mm) can be used. Leveraging the differential cavitation thresholds in fat vs. water-based tissue, the large focal zone transducer can make the fat reduction treatment very fast (>10 mL/min) while preserving the other tissue. Leveraging the differential threshold, the imaging guidance can be used to guide targeting, but is not required, which simplifies the setup. Histotripsy with a small focal zone and shallow focal depth can be used to treat subcutaneous fat close to the skin for precise fat reduction for face or body sculpturing.

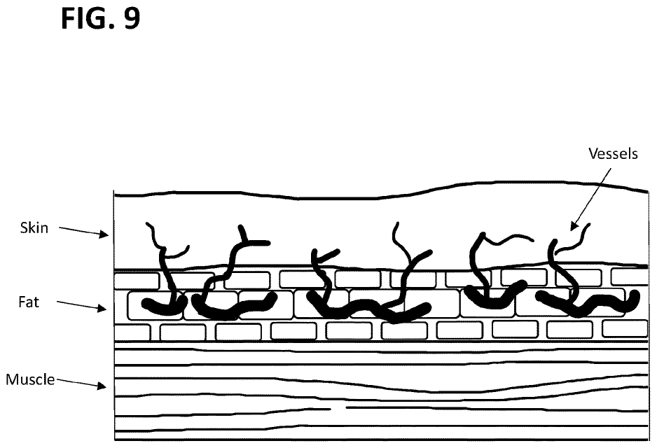

FIG. 9 is a cross-sectional view of human tissue including a skin layer, a fat layer, and a muscle layer. Vasculature including blood vessels is also shown. As described above, histotripsy pulses can be delivered into the subject to selectively ablate or liquefy fatty tissue without damaging surrounding tissues such as skin, muscle, or vessels. In some embodiments, the cycles can be configured to generate peak negative pressures above 14 MPa and below 26 MPa to liquefy the fat tissue without damaging or disrupting the skin tissues, muscle tissues, or vessels.

Alternatively, ACE signals measured in response to the transmitted pulses and cavitation can be processed or analyzed to determine the tissue type at the focal location. For example, it may not be known exactly how deep or thick the fat layer is. In one embodiment, test pulses can be transmitted at increasing depths within tissue, and the cavitation response can be monitored to determine the tissue type (e.g., skin, fat, muscle). The test points or focal locations can be mapped to determine the thickness or depth of each tissue type. Subsequent to this mapping, a treatment plan can be generated or adjusted to direct histotripsy pulses only within the fat region, including along the skin/fat border and the fat/muscle border. In this manner, personalized treatment plans can be generated that more efficiently target fatty tissue without targeting other sensitive tissues that aren't to be treated.

FIG. 10 shows a flowchart with a method for removing or reducing fat in a patient, comprising delivering histotripsy pulses to a target tissue with a peak negative pressure sufficient to liquefy fatty-based tissues and not water-based tissues (step 1002). As described above, the pulses can have a peak negative pressure between 14 MPa and under 26 MPa (step 1004). The pulses can be 1-cyle (step 1006). In some examples, the transducer can have a large focal volume (e.g., >5 mm) (step 1008) to reduce the treatment time.

### Mri-Based Real-Time Histotripsy Dose Monitoring

Periodic Diffusion weighted MRI (dMRI) treatment may also be used to monitor histotripsy doses spatially and temporally. In some embodiments, an MRI imaging system can be positioned around or near the subject such that MRI imaging can be applied periodically during the treatment. In some aspects, the entire histotripsy system is MRI compatible. In other aspects, the system (e.g., the transducer array) can be moved out of the imaging field of view when dMRI images are taken, but other aspects of the system can be MRI compatible (such as the coupling container).

The dMRI images can be evaluated to identify tissue changes within the target tissue volume in response to the cavitation, as reflected by changes to the apparent diffusion coefficient (ADC) in the dMRI images. In some embodiments, the ADC changes reflect the total dose delivered to the tissue, with increasing ADC indicating a higher dose received. The ADC changes, and therefore the dose of histotripsy delivered to the tissue, can be presented to the user. In some embodiments, the system can be configured to generate an ADC map with this data, which can be presented to the user or optionally overlaid onto other high-quality medical imaging of the target tissue volume (e.g., ultrasound, CT, MRI, etc.).

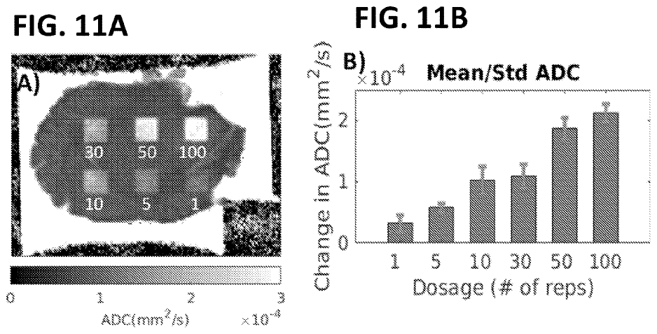

FIGS. 11A-11B show examples of histotripsy-generated damage in ex vivo brain, visible on dMRI. In FIG. 11A, 1 cm³ cubic lesions were generated in ex vivo bovine brain by electronically steering the focus to cover the target volume with varying number of pulses per focal location (1, 5, 10, 30, 50, and 100) for the histotripsy dose. As histotripsy mechanically breaks down the cellular members and extracellular matrix, the apparent diffusion coefficient (ADC) in the tissue changes to show on dMRI immediately post treatment. There is a trend of increasing ΔADC with increasing histotripsy dose, as shown in FIG. 11B. Therefore, an ADC map collected during histotripsy treatment periodically (e.g., for any preset time period such as every 6 seconds) can be overlaid on T1- or T2-weighted MRI to indicate histotripsy-induced damage both spatially and temporally. The ADC map can be used to identify one or more focal locations where complete cellular disruption has occurred. In some embodiments, an alert or indication can be provided to a user when complete cellular disruption has occurred at one or more focal locations in the target tissue.

Pre-treatment measurements (e.g., US or MRI elastography) can provide spatial tissue property measurements. It is possible to use these measurements to plan the spatial dose delivery.

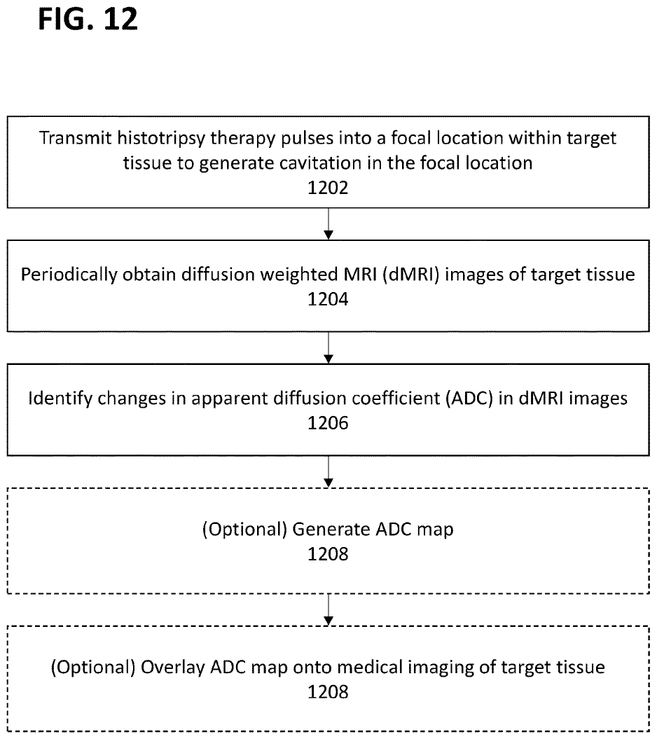

FIG. 12 a flowchart showing a method and algorithm which can be implemented in a histotripsy system of the present disclosure. The method can be a computer implemented method, or an algorithm or algorithms executed by a processor of a histotripsy system to cause the histotripsy system to perform the following operations: At step 1202, the method can include transmitting histotripsy therapy pulses into a focal location within target tissue with an ultrasound transducer array to generate cavitation in the focal location. In some aspects, transmitting the histotripsy therapy pulses can be for the purposes of testing the cavitation response at a particular focal location. In other embodiments, the pulses are transmitted with the intent to treat the focal location to cause complete cellular disruption. As described above, a plurality of transducer elements of the array can each transmit histotripsy pulses into the tissue. The ultrasound transducer can be mounted on a robotic arm and configured to deliver histotripsy pulses to one or more treatment or focal locations within a target tissue to generate cavitation at each focal location.

At step 1204, the system can periodically obtain diffusion weighted MRI (dMRI) images of the target tissue, such as with a MRI system coupled to or separate from the histotripsy system and in proximity to the patient. The system can obtain the dMRI images according to a preset time period (e.g., every 1 seconds, every 2 seconds, every 3, seconds, etc.). In some embodiments, the time period is less than 10 seconds between subsequent dMRI images).

At an operation 1206, the system can identify changes in the apparent diffusion coefficient (ADC) in the dMRI images. In some aspects, increasing ADC indicates increased dose delivered to the target tissue volume.

At an operation 1208, an optional ADC map can be generated which maps out the histotripsy dosages delivered at each focal location within a target tissue volume. At an operation 1208, the ADC map can optionally be overlaid onto other medical imaging of the target tissue volume, such as onto ultrasound imaging, MRI imaging, or CT imaging of the target tissue volume.

Histotripsy systems are provided that can use the ultrasound or MRI spatial and temporal feedback during histotripsy treatment, combined with pre-treatment tissue measurements, to predict and control the dose delivery and damage spatially and temporally to treat a specific tissue (e.g., white matter) while preserving another adjacent specific tissue within the target or homogeneously ablate the target tissue.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

1. A method of using a transmit-receive histotripsy system for histotripsy treatment monitoring, comprising the steps of:
transmitting high-voltage histotripsy therapy pulses into a focal location within a target tissue with transmit electronics and a histotripsy therapy transducer array to generate cavitation in the focal location;
receiving low-voltage acoustic cavitation emission (ACE) signals from the cavitation with receive electronics and the histotripsy therapy transducer array;
processing the received ACE signals to identify a cavitation lifespan of the cavitation at the focal location; and
identifying a plateau in the cavitation lifespan to determine that complete cellular disruption has occurred at the focal location.

2. The method of claim 1, further comprising generating a 3D map of cavitation produced by the transmitted pulses in real-time.

3. The method of claim 1, wherein identifying the plateau comprises identifying a time until a cavitation lifespan plateaus.

4. The method of claim 1, wherein identifying the plateau comprises identifying a number of pulses until a cavitation lifespan plateaus.

5. The method of claim 1, wherein identifying the plateau comprises identifying when the cavitation lifespan stops increasing.

6. The method of claim 1, further comprising automatically stopping transmitting high-voltage histotripsy therapy pulses when it has been determined that complete cellular disruption has occurred at the focal location.

7. The method of claim 1, further comprising providing an indication to a user that complete cellular disruption has occurred at the focal location.

8. The method of claim 1, wherein the indication comprises an audible alert.

9. The method of claim 1, wherein the indication comprises a visual alert.

10. A histotripsy system, comprising:
an ultrasound transducer array;
transmission electronics coupled to the ultrasound transducer array and configured to transmit one or more histotripsy pulses to one or more focal locations generate cavitation in a target tissue;
receive electronics configured to receive acoustic cavitation emissions (ACE) from the cavitation; and
one or more processors operatively coupled to the transmission and receive electronics, the one or more processors being configured to process the received ACE signals to identify a cavitation lifespan of the cavitation at the focal location and being further configured to identify a plateau in the cavitation lifespan to determine that complete cellular disruption has occurred at the focal location.

11. The system of claim 10, wherein the one or more processors are further configured to generate a 3D map of cavitation produced by the transmitted pulses in real-time.

12. The system of claim 11, further comprising a display configured to display the 3D map.

13. The method of claim 10, wherein the one or more processors are configured to identify the plateau by identifying a time until a cavitation lifespan plateaus.

14. The method of claim 10, wherein the one or more processors are configured to identify the plateau by identifying a number of pulses until a cavitation lifespan plateaus.

15. The method of claim 10, wherein the one or more processors are configured to identify the plateau by identifying when the cavitation lifespan stops increasing.

16. The method of claim 10, wherein the one or more processors are configured to stop transmitting high-voltage histotripsy therapy pulses when it has been determined that complete cellular disruption has occurred at the focal location.

17. The method of claim 10, wherein the one or more processors are configured to provide an indication to a user that complete cellular disruption has occurred at the focal location.

18. The method of claim 10, wherein the indication comprises an audible alert.

19. The method of claim 10, wherein the indication comprises a visual alert.

20. A method of monitoring histotripsy treatment, comprising the steps of:
transmitting high-voltage histotripsy therapy pulses into one or more focal locations within a target tissue with transmit electronics and a histotripsy therapy transducer array to generate cavitation in the focal location;
obtaining periodic diffusion weighted magnetic resonance imaging (dMRI) images of the one or more focal locations in the target tissue;
processing the dMRI images to identify apparent diffusion coefficients (ADC) in the one or more focal locations in the target tissue; and
generating an ADC map that conveys a dose of histotripsy therapy received at each of the one or more focal locations in the target tissue.

21. The method of claim 20, further comprising displaying the ADC map.

22. The method of claim 20, wherein increasing ADC in the target tissue indicates increased histotripsy dose received.

23. The method of claim 20, further comprising identifying complete cellular disruption at one or more focal locations in the target tissue.

24.-42. (canceled)
